(19) United States
(12) Patent Application Publication
(10) Pub. No.: US 2003/0053991 A1
(43) Pub. Date: Mar. 20, 2003

(54) Retinoic acid receptor beta-2, its agonists, and gene theraphy vectors for the treatment of neurological disorders

(76) Inventors: Alan John Kingsman, Robert Robinson Avenue (GB); Malcolm Maden, London (GB); Jonathan Patrick Thomas Corcoran, London (GB)

Correspondence Address: BKF JURGENSEN, 800 SILVERADO STREET, 2ND FLOOR, LA JOLLA, CA, 92037, US

(21) Appl. No.: 10/239,804

(22) PCT Filed: Mar. 30, 2001
(86) PCT No.: PCT/GB01/01478

(30) Foreign Application Priority Data
Oct. 4, 2000 (GB) 0024300.6

(51) Int. Cl. A61K 48/00; C12N 7/00; C12N 15/867; C12N 5/08

(52) U.S. Cl. 424/93.2; 435/235.1; 435/456; 435/320.1; 435/368

(57) ABSTRACT

The present invention relates to the use of RARβ2 and/or an agonist thereof in the preparation of a medicament to cause neurite development.

### FIELD OF THE INVENTION

The present invention relates to a factor relating to neurite growth. Furthermore, the invention relates to vectors capable of directing the expression of a factor relating to neurite growth.

### BACKGROUND TO THE INVENTION

The human peripheral and central nervous system consists of terminally differentiated cells which are not capable of directing neurite outgrowth or neurite regeneration.

It is desirable to cause neurite development, such as neurite outgrowth and/or neurite regeneration, for example in cases of nervous injuries such as spinal cord injuries or in diseases such as diabetes or neuropathies.

Nerve growth factor (NGF) is known to stimulate certain events such as neurite outgrowth. However, NGF is a relatively large molecule with a correspondingly high molecular weight. Moreover, NGF is susceptible to protease mediated degradation. Due to these and other considerations, NGF is difficult to administer. NGF is also relatively expensive to prepare. These are problems associated with the prior art.

### SUMMARY OF THE INVENTION

We have surprisingly found that it is possible to cause neurite development, such as neurite outgrowth and/or neurite regeneration, by using retinoic acid receptor β2 (RARβ2) and/or an agonist thereof. Moreover, it is surprisingly shown that RARβ2 can be delivered to non-dividing mammalian cells using vectors according to the invention.

### SUMMARY ASPECTS OF THE PRESENT INVENTION

The present invention is based on the surprising finding that it is possible to cause neurite development, such as neurite outgrowth and/or neurite regeneration, by using RARβ2 and/or an agonist thereof, and that RARβ2 may be introduced into neuronal cells using retroviral vectors based on lentiviral vectors.

Aspects of the present invention utilise this finding. For example it is possible to have a method that causes modulation of neurite development, such as neurite outgrowth and/or neurite regeneration, by using RARβ2 and/or a vector comprising same and/or an agonist thereof as explained herein.

### DETAILED ASPECTS OF THE PRESENT INVENTION

In one aspect, the present invention relates to a viral vector comprising a nucleic acid sequence encoding a receptor.

The viral vector may be based on or derived from a DNA virus, or an RNA virus (a retrovirus). Examples of such viral vectors include but are not limited to herpes viruses, adenoviruses, adeno-associated viruses, retroviruses, lentiviruses and other viruses. This is discussed in more detail below.

The receptor may be any eukaryotic receptor, such as a vertebrate receptor. Examples of such receptors include but are not limited to mammalian receptors, primate receptors and human receptors. This is explained more fully in the following section(s).

In another aspect, the present invention relates to a retroviral vector derived from a lentivirus genome comprising a nucleic acid sequence capable of directing the expression of a receptor.

In another aspect, the present invention relates to a viral vector comprising a nucleic acid sequence encoding the retinoic acid receptor β2 (RARβ2).

In another aspect, the present invention relates to a retroviral vector derived from a lentivirus genome comprising a nucleic acid sequence capable of directing the expression of the retinoic acid receptor β2 (RARβ2).

In another aspect, the present invention relates to a gene therapy vector comprising a nucleic acid sequence encoding a retinoic acid receptor β2. In a preferred aspect, delivery of the nucleic acid encoding the retinoic acid receptor β2 enables neurite growth.

In another aspect, the present invention relates to the use of a vector as described herein in the preparation of a medicament to cause neurite development.

In another aspect, the present invention relates to the use of a vector as described herein in the preparation of a medicament for the treatment of a neurological disorder.

In another aspect, the present invention relates to a method of treating a neurological disorder comprising administering a vector as described herein to a subject.

In another aspect, the present invention relates to a host cell when transduced by a vector as described herein.

In another aspect, the present invention relates to a pharmaceutical composition comprising a vector as described herein in admixture with a pharmaceutically acceptable carrier, diluent or excipient; wherein the pharmaceutical composition is for use to cause neurite development.

In another aspect, the present invention relates to the use of RARβ2 and/or an agonist thereof in the preparation of a medicament to cause neurite development.

The term ‘RARβ2’ as used herein may refer to the polypeptide translation product of the RARβ2 gene open reading frame (ORF), that is to say the actual receptor itself, or may refer to the nucleic acid ORF encoding said polypeptide, or may even occasionally refer to the RARβ2 gene itself. It will be apparent to the reader which of these entities, or combination of said entities, is referred to by the term ‘RARβ2’ from the particular context in which such term is used.

In the present invention the RARβ2 and/or an agonist can be termed a pharmaceutically active agent.

Neurites are well known structures which develop from various neuronal cell types. They appear as microscopic branch or comb-like structures or morphological projections from the surface of the cell from which they emanate. Examples of neurite outgrowth are shown in the accompanying figures, and in publications such as those referenced in (Maden 1998-review article), and are well known in the art.

The RARβ2 coding sequence (i.e. the RARβ2 gene) is used as described hereinbelow. The RARβ2 gene may be prepared by use of recombinant DNA techniques and/or by synthetic techniques. For example, it may be prepared using the PCR amplified gene fragment prepared as in the Examples section of this document using the primers etc. detailed therein, or it may be prepared according to any other suitable method known in the art.

In another aspect, the present invention relates to the use of RARβ2 and/or an agonist thereof in the preparation of a medicament to cause neurite development, wherein said agonist is retinoic acid (RA) and/or CD2019.

Retinoic acid is commercially available. CD2019 is a polycyclic heterocarbyl molecule which is a RARβ2 agonist having the structure as discussed herein and as shown in (Elmazar et al., (1996) Teratology vol. 53 pp158-167).

In another aspect, the present invention relates to the use of RARβ2 and/or an agonist thereof in the preparation of a medicament for the treatment of a neurological disorder.

In another aspect, the present invention relates to the use of RARβ2 and/or an agonist thereof in the preparation of a medicament for the treatment of a neurological disorder, wherein said neurological disorder comprises neurological injury.

In another aspect, the present invention relates to a method of treating a neurological disorder comprising administering a pharmacologically active amount of an RARβ2 receptor, and/or an agonist thereof.

In another aspect, the present invention relates to a method of treating a neurological disorder comprising administering a pharmacologically active amount of an RARβ2 receptor, and/or an agonist thereof, wherein said agonist is RA and/or CD2019.

In another aspect, the present invention relates to a method of treating a neurological disorder comprising administering a pharmacologically active amount of an RARβ2 receptor, and/or an agonist thereof, wherein said RARβ2 receptor is administered by an entity comprising a RARβ2 expression system.

In another aspect, the present invention relates to a method of causing neurite development in a subject, said method comprising providing a nucleic acid construct capable of directing the expression of at least part of a RARβ2 receptor, introducing said construct into one or more cells of said subject, and optionally administering a RARβ2 agonist, such as RA and/or CD2019, to said subject.

In a further aspect, the invention relates to an assay method for determining whether an agent is capable of modulating RARβ2 signalling, said method comprising providing neural cells, contacting said cells with said agent, and assessing the activity of the RARβ2 receptor, such as through the monitoring of neurite outgrowth.

Neural cells for use in the assay method of the invention may be any suitable neural cell line, whether stably maintained in culture, or primary cells derived from an animal directly. Preferably said cells will be embryonic mouse dorsal root ganglion (DRG) cells prepared as described hereinbelow.

In a further aspect, the invention relates to a process comprising the steps of (i) performing the assay for modulation of RARβ2 signalling described above, (ii) identifying one or more agents that are capable of modulating said RARβ2 signalling, and (iii) preparing a quantity of those one or more identified agents.

In a further aspect, the invention relates to a process comprising the steps of (i) performing the assay for modulation of RARβ2 signalling described above, (ii) identifying one or more agents that are capable of modulating said RARβ2 signalling, (iii) preparing a quantity of those one or more identified agents, and (iv) preparing a pharmaceutical composition comprising those one or more identified agents.

In a further aspect, the invention relates to a method of affecting the in vivo activity of RARβ2 with an agent, wherein the agent is capable of modulating RARβ2 signalling, for example capable of modulating RARβ2 signalling in an in vitro assay method as described above.

In a further aspect, the invention relates to the use of an agent in the preparation of a pharmaceutical composition for the treatment of a neurological disorder or injury, wherein the agent is capable of modulating RARβ2 signalling, for example capable of modulating RARβ2 signalling in an in vitro assay method as described above.

In a further aspect, the invention relates to a method of treating a subject with an agent, wherein the agent is capable of modulating RARβ2 signalling, for example capable of modulating RARβ2 signalling in an in vitro assay method as described above.

In a further aspect, the invention relates to a pharmaceutical composition comprising RARβ2 and/or an agonist thereof in admixture with a pharmaceutically acceptable carrier, diluent or excipient; wherein the pharmaceutical composition is for use to cause neurite development.

In a further aspect of the invention, there is provided a viral vector genome comprising nucleic acid sequence(s) capable of directing the expresion of a receptor. Preferably said vector genome comprises nucleic acid sequence(s) capable of directing the expression of at least part of the RARβ2 receptor.

In a further aspect of the invention, there is provided a retroviral vector genome comprising nucleic acid sequence(s) capable of directing the expresion of at least part of RARβ2, said genome containing a deleted gag gene from a lentivirus wherein the deletion in gag removes one or more nucleotides downstream of nucleotide 350 of the gag coding sequence. Preferably the deletion extends from nucleotide 350 to at least the C-terminus of the gagpol coding region. More preferably the deletion additionally removes nucleotide 300 of the gag coding region and most preferably the deletion retains only the first 150 nucleotides of the gag coding region. However even larger deletions of gag can also be used, for example the gag coding region may contain only the first 109 nucleotides of the gag coding region. It may also be possible for the gag coding region to contain only the first 2 nucleotides of the gag coding region. Preferably, said vector genome is capable of directing the expression of substantially all of the RARβ2 polypeptide.

Preferably, the vector of the present invention is based on or derived from a lentivirus. More preferably, the vector of the present invention is based on or derived from a non-primate lentivirus. In a highly preferred embodiment, the vector of the present invention is based on or derived from a non-primate lentivirus such as equine infectious anaemia virus (EIAV). This is discussed in more detail below.

Additional features of the lentiviral genome are included in the vector genome which are necessary for transduction of the target cell such as reverse transcription and integration. These are, at least, a portion of an LTR containing sequence from the R-region and U5 region, sequences adjacent to the 3′LTR which contain a polypurine tract (PPT) and a 3′LTR from the lentivirus or a hybrid LTR containing sequences from the lentivirus and other elements. Optionally, the retroviral genome may contain accessory genes derived from a retrovirus, such as, but not limited to, a rev gene, a tat gene, a vif gene, a nef gene, a vpr gene or an S2 gene. Additional components may be added such as introns, splice-donor sites, a rev responsive element (RRE), sequences called the cPPT containing the polymerase region (Stetor S R, Rausch J W, Guo M J, Burnham J P, Boone L R, Waring M J, Le Grice S F ‘Characterization of (+) strand initiation and termination sequences located at the center of the equine infectious anemia virus genome.’ Biochemistry. Mar. 23, 1999;38(12):3656-67), cloning sites and selectable marker genes.

Moreover, it has been demonstrated (eg. see WO 99/32646) that a lentivirus minimal vector system can be constructed which requires neither S2, Tat, env nor dUTPase for either vector production or for transduction of dividing and non-dividing cells. A lentivirus minimal vector system can also be constructed which requires neither S2, Tat, env, rev nor dUTPase for either vector production or for transduction of dividing and non-dividing cells.

Thus according to another aspect the lentivirus genome from which the vector is derived lacks one or more accessory genes.

The deletion of accessory genes is highly advantageous. Firstly, it permits vectors to be produced without the genes normally associated with disease in lentiviral (e.g. HIV) infections. In particular, tat and nef are associated with disease. Secondly, the deletion of accessory genes permits the vector to package more heterologous DNA. Thirdly, genes whose function is unknown, such as dUTPase and S2, may be omitted, thus reducing the risk of causing undesired effects.

In addition, we have shown that the leader sequence of the lentivirus genome is essential for high protein expression.

Therefore in a further aspect the lentivirus genome from which the vector is derived lacks the tat gene but includes the leader sequence between the end of the 5′ LTR and the ATG of gag.

These data further define a minimal essential set of functional components for an optimal lentiviral vector. A vector is provided with maximal genetic capacity and high titre, but without accessory genes that are either of unknown function (S2, UTPase), and therefore may present risk, or are analogues of HIV proteins that may be associated with AIDS (tat, rev).

It will be appreciated that the present invention provides a retroviral vector derived from a lentivirus genome comprising nucleic acid sequence capable of directing the expression of at least part of RARβ2 and (1) comprising a deleted gag gene wherein the deletion in gag removes one or more nucleotides downstream of nucleotide 350 of the gag coding sequence; (2) wherein one or more accessory genes are absent from the lentivirus genome; (3) wherein the lentivirus genome lacks the tat gene but includes the leader sequence between the end of the 5′ LTR and the ATG of gag; and combinations of (1), (2) and (3). In a preferred embodiment the retroviral vector comprises all of features (1) and (2) and (3).

A “non-primate” vector, as used herein, refers to a vector derived from a virus which does not primarily infect primates, especially humans. Thus, non-primate virus vectors include vectors which infect non-primate mammals, such as dogs, sheep and horses, reptiles, birds and insects.

A lentiviral or lentivirus vector, as used herein, is a vector which comprises at least one component part derived from a lentivirus. Preferably, that component part is involved in the biological mechanisms by which the vector infects cells, expresses genes or is replicated.

The lentivirus may be any member of the family of lentiviridae. Preferably the lentivirus is one which does not naturally infect a primate (‘non-primate lentivirus’). Such viruses may include a feline immunodeficiency virus (FIV), a bovine immunodeficiency virus (BIV), a caprine arthritis encephalitis virus (CAEV), a Maedi visna virus (MW) or an equine infectious anaemia virus (EIAV). Preferably the lentivirus is an EIAV. Equine infectious anaemia virus infects all equidae resulting in plasma viremia and thrombocytopenia (Clabough, et al. 1991. J Virol. 65:6242-51). Virus replication is thought to be controlled by the process of maturation of monocytes into macrophages.

EIAV has the simplest genomic structure of the lentiviruses. In addition to the gag, pol and env genes EIAV encodes three other genes: tat, rev, and S2. Tat acts as a transcriptional activator of the viral LTR (Derse and Newbold 1993 Virology. 194:530-6; Maury, et al 1994 Virology. 200:63242.) and Rev regulates and coordinates the expression of viral genes through rev-response elements (RRE) (Martarano et al 1994 J Virol. 68:3102-11.). The mechanisms of action of these two proteins are thought to be broadly similar to the analogous mechanisms in the primate viruses (Martano et al ibid). The function of S2 is unknown. In addition, an EIAV protein, Ttm, has been identified that is encoded by the first exon of tat spliced to the env coding sequence at the start of the transmembrane protein.

In addition to protease, reverse transcriptase and integrase lentiviruses contain a fourth pol gene product which codes for a dUTPase. This may play a role in the ability of these lentiviruses to infect certain non-dividing cell types.

The viral RNA in aspect(s) of the invention is transcribed from a promoter, which may be of viral or non-viral origin, but which is capable of directing expression in a eukaryotic cell such as a mammalian cell. Optionally an enhancer is added, either upstream of the promoter or downstream. The RNA transcript is terminated at a polyadenylation site which may be the one provided in the lentiviral 3′ LTR or a different polyadenylation signal.

Thus the present invention provides a DNA transcription unit comprising a promoter and optionally an enhancer capable of directing expression of a retroviral vector genome.

Transcription units as described herein comprise regions of nucleic acid containing sequences capable of being transcribed. Thus, sequences encoding mRNA, tRNA and rRNA are included within this definition. The sequences may be in the sense or antisense orientation with respect to the promoter. Antisense constructs can be used to inhibit the expression of a gene in a cell according to well-known techniques. Nucleic acids may be, for example, ribonucleic acid (RNA) or deoxyribonucleic acid (DNA) or analogues thereof. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements. Nucleic acids may comprise cDNA or genomic DNA (which may contain introns).

In another aspect, the present invention relates to a retroviral vector derived from a lentivirus genome comprising a nucleic acid sequence capable of directing the expression of at least part of RARβ2 and comprising a deleted gag gene wherein the deletion in gag removes one or more nucleotides downstream of nucleotide 350 of the gag coding sequence.

In another aspect, the present invention relates to a retroviral vector as described herein, wherein the deletion extends from nucleotide 350 to at least the C-terminus of the gag-pol coding region.

In another aspect, the present invention relates to a retroviral vector as described herein, wherein the deletion additionally removes nucleotide 300 of the gag coding region.

In another aspect, the present invention relates to a retroviral vector as described herein, wherein the deletion retains the first 150 nucleotides of the gag coding region. In another aspect, the present invention relates to a retroviral vector as described herein, wherein the deletion retains the first 109 nucleotides of the gag coding region.

In another aspect, the present invention relates to a retroviral vector as described herein, wherein the deletion retains only the first 2 nucleotides of the gag coding region.

In another aspect, the present invention relates to a retroviral vector as described herein, wherein the deletion is of the entire gag coding region.

In another aspect, the present invention relates to a retroviral vector derived from a lentivirus genome wherein one or more accessory genes are absent from the lentivirus genome.

In another aspect, the present invention relates to a retroviral vector as described herein, wherein the accessory genes are selected from dUTPase, S2, rev and tat.

In another aspect, the present invention relates to a retroviral vector derived from a lentivirus genome such as EIAV wherein the lentivirus genome lacks the tat gene but includes the leader sequences between the end of the 5′ LTR and the ATG of gag.

In another aspect, the present invention relates to a retroviral vector as described herein, which comprises at least one component from an equine lentivirus.

In another aspect, the present invention relates to a retroviral vector as described herein, wherein the equine lentivirus is EIAV.

In another aspect, the present invention relates to a retroviral vector as described herein, wherein the retroviral vector is substantially derived from EIAV.

In another aspect, the present invention relates to a method comprising transfecting or transducing a cell with a retroviral vector as described herein.

In another aspect, the present invention relates to a delivery system in the form of a retroviral vector as described herein.

In another aspect, the present invention relates to a cell transfected or transduced with a retroviral vector as described herein.

In another aspect, the present invention relates to use of a retroviral vector as described herein.

In another aspect, the present invention relates to use of a gene therapy vector as described herein.

In another aspect, the invention relates to the use of lentiviral gene therapy vectors for the delivery of retinoic acid receptor β2 to the peripheral and central nervous systems.

In another aspect, the present invention relates to a gene therapy vector comprising a nucleic acid sequence encoding a retinoic acid receptor β2. In a preferred aspect, delivery of the nucleic acid encoding the retinoic acid receptor β2 enables neurite growth.

In another aspect, the invention relates to EIAV gene therapy vectors configured to express retinoic acid receptor β2 (RARβ2).

In another aspect, the invention relates to methods for producing expression of RARβ2 in adult mammalian (such as human) spinal cord. Expression of RARβ2 in adult spinal cord is shown to stimulate neurite outgrowth and regeneration. Thus, in a preferred aspect, the invention relates to methods for stimulation of neurite outgrowth and/or regeneration in mammalian neuronal cells.

As used herein, the term ‘adult’ is used to mean non-foetal and/or non-embryonic. The term thus includes adults per se, as well as including young such as children and/or pups or other such infants. Thus, the term ‘adult’ as used herein may be understood to include any ‘post-natal’ ie. post-birth organism.

In another aspect, the invention relates to a differential expression screening method for identifying genes involved in a cellular process which method comprises comparing gene expression in: a first cell of interest; and a second cell of interest which cell comprises altered levels, relative to physiological levels, of a biological molecule due to the introduction into the second cell of a heterologous nucleic acid encoding at least part of RARβ2; and identifying gene products whose expression differs. Preferably, said heterologous nucleic acid encodes substantially all of RARβ2. Optionally, retinoic acid or an analogue thereof may also be present in the cellular environment of one or preferably both cells of interest. This method or a variant thereof may be advantageously applied to comparison of non-dividing neuronal cells with a different sample of the same cells which have been induced to exhibit neurite outgrowth, such as via transduction with a vector delivering RARβ2, or via other techniques discussed herein.

For ease of reference, these and further aspects of the present invention are now discussed under appropriate section headings. However, the teachings under each section are not necessarily limited to each particular section.

Preferable Aspects

In a preferred aspect, the administration of a nucleic acid construct capable of directing the expression of RARβ2 will be accompanied by the administration of a RARβ2 agonist such as RA, or preferably CD2019 (or a mimetic thereof.

Preferably said agonist will be to some degree selective for the RARβ2 receptor. Preferably said agonist will not significantly affect the RARα receptor. Preferably said agonist will not significantly affect the RARγ receptor. More preferably said agonist will not significantly affect the RARα receptor or the RARγ receptor. Even more preferably, said agonist will exhibit a high degree of selectivity for the RARβ2 receptor.

In a preferred aspect, the administration of a nucleic acid construct capable of directing the expression of RARβ2 will be accomplished using a vector, preferably a viral vector, more preferably a retroviral vector. In a highly preferred embodiment, the administration of a nucleic acid construct capable of directing the expression of RARβ2 will be accomplished using a retroviral vector capable of infecting non-dividing mammalian cells such as neural cells. This retroviral vector will preferably be derived from a lentiviral vector (preferably a non-primate lentiviral vector as discussed above), more preferably said vector will be derived from an equine infectious anaemia virus (EIAV). In a highly preferred aspect, said EIAV-derived vector will be a pseudotyped particle, such as VSV-G pseudotyped, or Rabies G pseudotyped.

Advantages

The present invention is advantageous because RARβ2 and/or an agonist thereof can cause modulation of neural cell development.

It is also an advantage of the present invention that administration of NGF to a subject is avoided.

It is also an advantage of the present invention that it enables neurite outgrowth to be promoted in adult neural tissue.

It is also an advantage of the present invention that it enables RARβ2 to be introduced into non-dividing mammalian cells such as neuronal cells.

It is also an advantage of the present invention that the receptor may be delivered to cells whose environment comprises endogenous levels of agonist of the receptor, such as retinoic acid (RA).

Retinoids

Retinoids are a family of molecules derived from vitamin A and include the biologically active metabolite, retinoic acid (RA). The cellular effects of RA are mediated through the action of two classes of receptors, the retinoic acid receptors (RARS) which are activated both by all-trans-RA (tRA) and 9-cis-RA (9-cis-RA), and the retinoid X receptors (RXRs), which are activated only by 9-cis-RA (Kastner et al., 1994; Kleiwer et al., 1994). The receptors are of three major subtypes, α, β and γ, of which there are multiple isoforms due to alternative splicing and differential promoter usage (Leid et al.). The RARs mediate gene expression by forming heterodimers with the RXRs, whilst the RXRs can mediate gene expression as homodimers or by forming heterodimers with a variety of orphan receptors (Mangelsdorf & Evans, 1995). Many studies on a variety of embryonic neuronal types have shown that RA can stimulate both neurite number and length (review, Maden, 1998), as, indeed, can the neurotrophins (Campenot, 1977; Lindsay, 1988; Tuttle and Mathew, 1995). The neurotrophins are a family of growth factors that are required for the survival of a variety of neurons of primary sensory neurons in the developing peripheral nervous system (Snider, 1994). One of the earliest genes induced by NGF in PC12 cells is the orphan receptor NGFI-B (NURR1) (Millbrandt, 1989). This suggests that the growth factor and retinoid mediated pathway in developing neurons can interact.

Background teachings on these aspects have been presented by Victor A. McKusick et al on http://www.ncbi.nim.nih.gov/Omim. The following information has been extracted from that source.

Three retinoic acid receptors, alpha, beta, and gamma, are members of the nuclear receptor superfamily. Retinoic acid was the first morphogen described in vertebrates. The RARA and RARB genes are more homologous to those of the 2 closely related thyroid hormone receptors THRA and THRB, located on chromosomes 17 and 3, respectively, than to any other members of the nuclear receptor family. These observations suggest that the thyroid hormone and retinoic acid receptors evolved by gene, and possibly chromosome, duplications from a common ancestor which itself diverged rather early in evolution from the common ancestor of the steroid receptor group of the family. The RARB gene, formerly symbolized HAP, maps to 3p24 by somatic cell hybridization and in situ hybridization.

Benbrook et al. (1988) showed a predominant distribution in epithelial tissues and therefore used the designation RAR(epsilon). By in situ hybridization, Mattei et al. (1988) assigned the RARB gene to 3p24. Using deletion mapping, de The et al. (1990) identified a 27-bp fragment located 59-bp upstream of the transcriptional start, which confers retinoic acid responsiveness on the herpesvirus thymidine kinase promoter. They found indications that both alpha and beta receptors act through the same DNA sequence. Mattei et al. (1991) assigned the corresponding gene to chromosome 14, band A, in the mouse, and to chromosome 15 in the rat.

Nadeau et al. (1992) confirmed assignment of the mouse homolog to the centromeric portion of chromosome 14.

From a comparison of a hepatitis-B virus (HBV) integration site present in a particular human hepatocellular carcinoma (HCC) with the corresponding unoccupied site in the nontumorous tissue of the same liver, Dejean et al. (1986) found that HBV integration placed the viral sequence next to a liver cell sequence that bears a striking resemblance to both an oncogene, ERBA, and the supposed DNA-binding domain of the human glucocorticoid receptor and estrogen receptor genes.

Dejean et al. (1986) suggested that this gene, usually silent or transcribed at a very low level in normal hepatocytes, becomes inappropriately expressed as a consequence of HBV integration, thus contributing to the cell transformation.

By means of a panel of rodent-human somatic cell hybrid DNAs, Dejean et al. (1986) localized the gene to chromosome 3. Further studies by de The et al. (1987) suggested that the HAP gene product may be a novel ligand-responsive regulatory protein whose inappropriate expression in liver is related to hepatocellular carcinogenesis. Brand et al. (1988) showed that the novel protein called HAP (for HBV-activated protein) is a retinoic acid receptor. They referred to this receptor as the beta type (RARB) and mapped it to 3p25-p21.

Lotan et al. (1995) found that the expression of RARB mRNA is selectively lost in premalignant oral lesions and can be restored by treatment with isotretinoin. Restoration of the expression of RARB mRNA was associated with a clinical response.

RARB, RARG, RXRB, and RXRG are expressed in the striatum. To study the effect of these genes on locomotion, Kreczel et al. (1998) developed single and double knockout mice and analyzed their locomotor skills by open field and rotarod testing. RARB-RXRB, RARB-RXRG, and RXRB-RXRG double null mutant mice, but not the corresponding single null mutants, exhibited reductions in forward locomotion when compared with wildtype littermates. Forty percent of the RARB-RXRB null mutants showed backward locomotion. Rotarod test performance was impaired for RARB, RARB-RXRB, RARB-RXRG, and RXRB-RXRG mice. In contrast, RARA, RARG, RARA-RXRG, and RARG-RXRG null mice showed no defects in locomotion, even though both RARA and RARG are also expressed in the striatum. The morphology, development, and function of skeletal muscle, peripheral nerves, and spinal cord were normal in all single and double null mutants, as were balance reflexes. These results suggested to Kreczel et al. (1998) that RARB, RXRB, and RXRG are involved specifically in the control of locomotor behaviors, and that heterodimers of RARB with either RXRB or RXRG are the functional receptor units, such that RXRB and RXRG are functionally redundant.

Kreczel et al. (1998)studied the expression of D1 and D2 dopamine receptors (D1R and D2R), the most abundant dopamine receptors in the striatum, in these mutant mice. RARB-RXRB, RARB-RXRG, and RXRB-RXRG double null mutants, but not RARB or RXRG single mutants, exhibited 40% and 30% reduction in whole-striatal DIR and D2R transcripts, respectively, when compared with wildtype controls.

The reduction was mostly in the medioventral regions of the striatum, including the shell and core of the nucleus accumbens, and the mediodorsal part of the caudate putamen. The reduction was not due to loss of D2R-expressing neurons; no increase in apoptosis was noted. The histology of the striatum was normal.

The characterization of a retinoic acid response element in the D2R promoter by Samad et al. (1997) led Kreczel et al. (1998) to suggest that the reduction in D2R and D2R expression occurs on a transcriptional level. The RARB-RXRB, RARB-RXRG, and RXRB-RXRG double null mutants did not exhibit the normal increase in locomotion induced by cocaine, mimicking the phenotype of D1R-null mice.

Taken together, these results indicated to Kreczel et al. (1998) that retinoids are involved in controlling the function of the dopaminergic mesolimbic pathway and suggested that defects in retinoic acid signaling may contribute to neurological disorders.

AGONISTS

The agonist of the present invention may be any suitable RARβ2 agonist. Preferably, said agonist of RARβ2 is capable of activating RARβ2 in a transactivation assay.

The agonist may be an organic compound or other chemical. The agonist can be an amino acid sequence or a chemical derivative thereof, or a combination thereof. The agent may even be a nucleotide sequence—which may be a sense sequence or an anti-sense sequence. The agent may even be an antibody.

Typically, the agonist will be an organic compound. Typically the organic compound will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group, such as a non-fused polycyclic group. For some applications, the agonist comprises at least the one of said cyclic groups linked to another hydrocarbyl group.

Specific Agonists

An example of a specific agonist according to the present invention is retinoic acid (RA). Both common forms of retinoic acid (either all-trans retinoic acid (tRA), or 9-cis-RA) are agonists of RARβ2.

CD2019 is a RARβ2 agonist having the structure as discussed herein and as shown in (Elmazar et al., (1996) Teratology vol. 53 pp158-167). This and other agonists are also discussed in (Beard and Chandraratna p.194; Johnson et al., 1996). The structure of CD2019 is presented as Formula I in the attached figures.

An alternative RARβ2 agonist is presented as Formula II in the attached figures.

The present invention also encompasses mimetics or bioisosteres of the formulae of Formula I and/or Formula II.

Preferably the agonist useful according to the present invention is selective for RARβ2.

Assay to Determine RARβ2 Agonism

Examples of agonists according to the present invention may be identified and/or verified by using an assay to determine RARβ2 agonism.

Hence, the present invention also encompasses (i) detemining if a candidate agent is capable of acting as a RARβ2 agonist; (ii) if said candidate agent is capable of acting as a RARβ2 agonist then delivering said agent to a subject and in such an amount to cause neurite development.

Assay

Any one or more of appropriate targets—such as an amino acid sequence and/or nucleotide sequence—may be used for identifying an agent capable of modulating RARβ2 in any of a variety of drug screening techniques. The target employed in such a test may be free in solution, affixed to a solid support, borne on a cell surface, or located intracellularly. The abolition of target activity or the formation of binding complexes between the target and the agent being tested may be measured.

The assay of the present invention may be a screen, whereby a number of agents are tested. In one aspect, the assay method of the present invention is a high through put screen.

Techniques for drug screening may be based on the method described in Geysen, European Patent Application 84/03564, published on Sep. 13, 1984. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO-A-84/03564.

It is expected that the assay methods of the present invention will be suitable for both small and large-scale screening of test compounds as well as in quantitative assays.

In one preferred aspect, the present invention relates to a method of identifying agents that selectively modulate RARβ2.

In a preferred aspect, the assay of the present invention utilises cells that display RARβ2 on their surface. These cells may be isolated from a subject possessing such cells. However, preferably, the cells are prepared by transfecting cells so that upon transfect those cells display on their surface RARβ2.

Another example of an assay that may be used is described in WO-A-9849271, which concerns an immortalised human terato-carcinoma CNS neuronal cell line, which is said to have a high level of neuronal differentiation and is useful in detecting compounds which bind to RARβ2.

In another aspect, the invention relates to the use of a vector capable of directing the expression of RARβ2 to produce cell(s) for use in agonist/antagonist assays. For example, in another aspect, the invention relates to an assay comprising neuronal cell(s), said cells comprising an EIAV-derived vector capable of directing the expression of RARβ2 in said cell(s).

Reporters

A wide variety of reporters may be used in the assay methods (as well as screens) of the present invention with preferred reporters providing conveniently detectable signals (eg. by spectroscopy). By way of example, a reporter gene may encode an enzyme which catalyses a reaction which alters light absorption properties.

Other protocols include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes may even be used. These and other assays are described, among other places, in Hampton R et al (1990, Serological Methods, A Laboratory Manual, APS Press, St Paul Minn.) and Maddox D E et al (1983, J Exp Med 15 8:121 1).

Examples of reporter molecules include but are not limited to (galactosidase, invertase, green fluorescent protein, luciferase, chloramphenicol, acetyltransferase, (glucuronidase, exo-glucanase and glucoamylase. Alternatively, radiolabelled or fluorescent tag-labelled nucleotides can be incorporated into nascent transcripts which are then identified when bound to oligonucleotide probes.

By way of further examples, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 2,277,437; U.S. Pat. No. 4,275,149 and U.S. Pat. No. 4,366,241.

Differential Expression Screening Techniques

Genes encode gene products, mainly polypeptides but also RNAs, that are involved in a huge variety of cellular processes. The technique of differential expression screening is based on the idea that by comparing expression under two sets of conditions, genes whose expression varies between those two conditions can be identified and their function related back to the differences between those conditions. For example, genes involved in a pathway responsive to mitogens such as plate-derived growth factor (PDGF) can be identified by comparing gene expression in cells exposed to PDGF versus gene expression in cells not exposed to PDGF. Thus the term “differential expression screening” as used herein means comparing gene expression between two cells under different conditions or two different cells under the same or different conditions, with the aim of identifying gene products that differ in their levels of expression between the two cells.

The differences in gene expression may be measured using a variety of techniques. The first main type of technique is based on the measurement of nucleic acids and is termed herein as “genomic or cDNA techniques”. A useful review is provided in Kozian and Kirschbaum (1999). The second main type of technique is based on the measurement of cellular protein content and is termed herein as “proteomic techniques”.

Genomic or cDNA Techniques

One method well known in the art is subtractive cDNA hybridisation. This technique involves hybridising a population of mRNAs from one cell (e.g. a control cell) with a population of cDNAs made from the mRNA of another cell (e.g. a cell exposed to PDGF). This step will remove all sequences from the cDNA preparation that are common to both cells. The cDNAs derived from mRNAs whose expression is upregulated in the cell exposed to PDGF will not have a corresponding mRNA from the control with which to hybridise and can be isolated. Typically, the cDNAs are also hybridised with mRNA from the same cell to confirm that they represent coding sequences. This procedure is described in detail in WO90/11361 where mRNA from cells from the roots of plants treated with a chemical, N-(amincarbonyl)-2-chlorobenzenesulphonamide, were used to produce a cDNA library that was then hybridised with mRNA from untreated root cells. The procedure identified a number of genes whose expression was upregulated by the chemical.

The polymerase chain reaction (PCR) has led to the development of a number of other methods. RT-PCR differential display was first described by Liang and Pardee (1992). This technique involves the use of oligo-dT primers and random 5′ oligonucleotide 10-mers to carry out PCR on reverse-transcribed RNA from different cell populations. PCR is often carried out using a radiolabelled nucleotide so that the products can be visualised after gel electrophoresis and autoradiography. Wilkinson et al. (1995) used PCR differential display to identify five mRNAs that are upregulated in strawberry fruit during ripening. A review of differential display RT-PCR (also known as differential display of mRNA) is provided in Zhang et a/. (1998) and a recent improvement using ‘long distance’ PCR is described in Zhao et al. (1999).

Another technique is termed cDNA library screening. A review of this technique and the other two differential expression screening techniques mentioned above is provided in Maser and Calvet (1995).

Differential display competitive PCR is a fairly recent innovation that has been successfully used to study changes in global gene expression in situations where only a few genes change expression levels, such as exposure of MCF17 cell to oestradiol, and in more complex situations such as neuronal differentiation of human NTERA2 cells (Jorgensen et al., 1999).

A further PCR based technique is representational difference analysis (RDA)—see Kozian and Kirschbaum (1999) for review and references therein. Also reviewed in see Kozian and Kirschbaum (1999) is a technique termed serial analysis of gene expression.

The actual identification of gene products whose expression differs between the two cell populations can be carried out in a number of ways. Subtractive methods will inherently identify gene products whose expression differs since gene products whose expression is the same are eliminated from the sample. Other methods include simply comparing the expression products from one cell with the expression products from another and looking for any differences (with PCR-based techniques, the number of products in each sample can be limited to a reasonable size), optionally with the aid of a computer program. For example using a PCR-based technique a visual comparison of bands present in different lanes allows the identification of bands unique to one lane. These bands can be cut out of the gel and subsequently analysed.

The advent of DNA chip technology, allows comparisons to be conveniently conducted by the use of microarrays (see Kozian and Kirschbaum, 1999 for review and references therein). Typically, arrays are generated using cDNAs (including ESTs), PCR products, cloned DNA and synthetic oligonucleotides that are fixed to a substrate such as nylon filters, glass slides or silicon chips. To determine differences in gene expression, labelled cDNAs or PCR products are hybridised to the array and the hybridisation patterns compared. The use of fluorescently labelled probes allows two different cell populations to be applied simultaneously to one chip and the results measured at different wavelengths A microarray-based differential expression screening technique is described in U.S. Pat. No. 5,800,992.

Proteomic Techniques

Proteomics is the study of proteins properties on a large scale to obtain a global, integrated view of disease processes, cellular processes and networks at the protein level. A review of techniques used in proteomics is given in Blackstock and Weir (1999)—see also references provided therein. The methods of the present invention are mainly concerned with expression proteomics, the study of global changes in protein expression in cells using electrophoretic techniques and image analysis to resolve proteins. Whereas nucleic acid analysis emphasises the message, proteomics is more concerned with the product. The two approaches are sometimes complementary since proteomic techniques may be useful in detecting changes in polypeptide levels due to changes in protein stability rather than mRNA levels.

A well known and ubiquitous technique used in the field of proteomics involves measuring the polypeptide content of a cell using 2D polyacrylamide gel electrophoresis (PAGE) and comparing this with the polypeptide content of another cell. The results of electrophoresis are typically a gel visualised with a dye such as silver stain or Coomassie-blue, or an autoradiograph produced from the gel, all with spots corresponding to individual proteins. Fluorescent dyes are also available.

The aim is therefore to identify spots that differ between the two gels/autoradiographs, i.e. missing from one, reduced in intensity or increased in intensity. Thus in the case of proteomics, comparing gene expression simply involves comparing the protein profile from one cell with the protein profile from another. Commercial software packages are available for automated spot detection.

Spots of interest may be excised from gels and the proteins identified using techniques such as matrix-assisted-laser-desorption-ionisation-time-of-flight (MALDI-TOF) mass spectrometry and electrospray.

It may be desirable to perform some measure of prefractionation, such as centrifugation or free-flow electrophoresis to improve the identification of low abundance proteins. Special procedures have also been developed for basic proteins, membrane proteins and other poorly soluble proteins (Rabilloud et al., 1997).

The above discussion provides a description of prior art methods available to the skilled person for performing differential expression screening of two or more cell populations in a general sense. However, the present invention is distinguished from these prior art methods in that a further step is required, namely that the levels of an endogenous biological molecule in a cell are altered by the experimenter, so that the levels of gene products that are affected by the molecule become more responsive to cellular perturbations such as signalling events. In other words, the object is to amplify and/or increase the signal to noise ratio of the differential response normally obtained so as to increase the likelihood of detecting gene products whose levels in a cell are low and/or whose expression normally changes by only a small amount.

By way of an example, the transcription factor HIF-1α is responsive to intracellular oxygen levels. Decreases in oxygen levels increase HIF-1α activity and lead to increased transcription from genes comprising a hypoxia responsive element (HRE). If the levels of HIF-1α in the cell are raised artificially, for example by infecting cells with a viral vector that directs expression of HIF-1α, then you would expect to see an increase in the transcriptional response mediated by HIF-1α. Consequently, changes in the expression of genes whose expression is sensitive to the HIF-1α mediated hypoxic response should be greater than in normal cells expressing physiological levels of HIF-1α.

Biological Molecules

The biological molecule can be any compound that is found in cells as a result of anabolic or catabolic processes within a cell or as a result of uptake from the extracellular environment, by whatever means. The term “biological molecule” means that the molecule has activity in a biological sense. Preferably the biological molecule is synthesised within the cell, i.e. is endogenous to that cell, or in the case of multicellular organisms, also within any of the cells of the organism.

Examples of biological molecules will therefore include proteins, nucleic acids, carbohydrates, lipids, steroids, co-factors, prosthetic groups (such as haem), inorganic molecules, ions (such as Ca²⁺), inositides. Where appropriate, precursors, monomeric, oligomeric and polymeric forms, and breakdown products of the above are also included.

Example of polypeptides include enzymes, transcription factors, hormones, structural components of cells and receptors including membrane bound receptors.

Preferably, the biological molecule is known to be involved in the cellular process of interest.

In one embodiment of the invention, the biological molecule is responsive to a signal, which may be an externally applied signal such as an environmental signal, for example redox stress, the binding of an extracellular ligand to a cell surface receptor leading to a cellular response mediated by a signal transduction signal. Alternatively, the signal may be an internally applied signal such as an increase in kinase activity due to failing levels of a cell metabolite.

The levels of the biological molecule may be altered directly or indirectly. Direct alteration may be achieved by, for example, causing cells to take up the molecule by incubating cells in a medium containing higher than physiological levels of the molecule. Other methods include vesicle-mediated delivery and microinjection. In the case of nucleic acids and polypeptides, the level of the biological molecule in the cell may be raised by the introduction of a heterologous nucleic acid into the cell which directs the expression of the nucleic acid or polypeptide.

The term “heterologous nucleic acid” in the present context means that the nucleic acid is not present in its natural context i.e. the cell has been modified so as to contain the nucleic acid which would otherwise not be present in the form in which it is introduced. For example, the nucleic acid may be extrachromosomal. The nucleic acid may also be integrated into the genome by viral transduction or homologous recombination. Nonetheless, part of all of the heterologous nucleic may be identical to a corresponding genomic sequence since the introduction of additional copies of a gene is a convenient means for increasing the levels of expression of that gene.

Indirect means for altering the levels of the biological molecule are numerous and include increasing the levels of an inhibitory or stimulatory molecule using the methods described above. Inhibitory molecules include antisense nucleic acids, ribozyme or an EGS directed against the mRNA encoding the biological molecule, a transdominant negative mutant directed against the biological molecule, transcription factors, enzyme inhibitors, and intracellular antibodies such as scFvs. Stimulatory molecules include enzyme activators, transcriptional activators. Thus cells may be manipulated in a number of ways such that ultimately the levels of the biological molecule are altered. Reduced expression may be achieved by expressing an anti-sense RNA,

The levels of the biological molecule are altered relative to physiological levels. Thus they may be enhanced or reduced. The term “relative to physiological levels” means relative to the concentration or activity of the biological molecule typically present in the cell under normal physiological conditions prior to manipulation of those levels. Thus the intention is that by deliberate means, the activity of the biological molecule is altered above or below that which is found in the cell under a range of normal physiological conditions. “Physiological conditions” includes the conditions normally found in vivo and the conditions normally used in vitro to culture the cells.

By way of an example, the activity or concentration may be increase or decreased 5-fold, 10-fold, 20-fold, 50-fold or 100-fold compared to the normal physiological activity or concentration found in the cell prior to introducing, for example, the heterologous nucleic acid.

Where, as in a preferred embodiment of the invention, the levels of the biological molecule are altered by the introduction of a heterologous nucleic acid, typically a nucleic acid that directs expression of a polypeptide, the heterologous nucleic acid will comprise a coding sequences operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Control sequences operably linked to sequences encoding the protein of the invention include promoters/enhancers and other expression regulation signals. These control sequences may be selected to be compatible with the host cell in which the expression vector is designed to be used. The term promoter is well known in the art and encompasses nucleic acid regions ranging in size and complexity from minimal promoters to promoters including upstream elements and enhancers.

The promoter is typically selected from promoters which are functional in mammalian, cells, although promoters functional in prokaryotic cells or other eukaryotic cells may be used where appropriate. Thus, the promoter is typically derived from promoter sequences of viral or eukaryotic genes. For example, it may be a promoter derived from the genome of a cell in which expression is to occur. Eukaryotic promoters, may be promoters that function in a ubiquitous manner (such as promoters of α-actin, β-actin, tubulin) or, alternatively, a tissue-specific manner (such as promoters of the genes for pyruvate kinase). Tissue-specific promoters specific for particular cells may be used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may be advantageous for the promoters to be inducible so that the levels of expression from the heterologous nucleic acid can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Suitable vectors include plasmids, artificial chromosomes and viral vectors. Viral vectors include DNA virus vectors, RNA virus vectors (ie. retroviral vectors), such as lentiviruses, adenoviral vectors, adeno-associated vectors and herpes simplex viral vectors. Vectors/polynucleotides may introduced into suitable host cells using a variety of techniques known in the art, such as transfection, transformation, electroporation, infection with recombinant viral vectors such as retroviruses, herpes simplex viruses and adenoviruses, direct injection of nucleic acids and biolistic transformation. It is particularly preferred to use recombinant viral vector-mediated techniques.

A cell of interest can be any cell, for example a prokaryotic cell, a yeast cell, a plant cell or an animal cell, such as an insect cell or a mammalian cell, including a human cell. In the case of cells from multicellular organism, cells may be primary cells or immortalised cell lines. Although cells are frequently referred to in the singular, in general cells will be part of a cell population.

In certain aspects of the invention, a comparison is required between gene expression in at least two distinct cells. Typically the first of the two or more cells is termed a reference cell. In a preferred embodiment, the cells to be used in the comparison are substantially identical in all respects. For example they may both be cells of the same cell line or obtained from the same tissue in an organism. One or both of the cells may then be manipulated so that they comprise altered levels, relative to physiological levels, of the biological molecule as described above. In one embodiment, the first cell is unaltered and the second cell altered. This is particularly preferred since it should result in an improved signal to noise ratio. In a highly preferred embodiment, the first cell is unaltered, and the second cell comprises RARβ2 according to the present invention. Preferably, the cells are mammalian neuronal cells.

Nonetheless, it is not necessary that the cells used as the starting point of the investigation be substantially identical. For example, in one aspect of the invention, genes involved in disease processes may be investigated using cells from a diseased organism, such as a mammalian patient. These may be compared with cells from a normal organism or similar cells from the same or a different diseased individual. Where cells from a normal organism and a diseased organism are used, generally the normal cells correspond to the first cell of interest and the diseased cells correspond to the second cell of interest. Consequently, at least the diseased cells are modified as described above in so that comprises altered levels of the biological molecule.

In another embodiment, one cell is a cell comprising a mutant gene whereas the other cell comprises a wild-type version of the same gene.

Another possibility is that the cells are from different tissues or from different stages in development or differentiation, for example as affected by the presence or absence of RARβ2, and/or retinoic acid or derivatives thereof.

The present invention provides a number of improved methods for identifying genes by differential expression screening techniques.

In another aspect, a method is provided for identifying genes involved in a cellular process. Essentially one of the cells is manipulated so that the levels within that cell of a biological molecule involved in the cellular process are altered. Preferably, this process is neurite outgrowth and/or neural regeneration as effected by the action of retinoic acid through RARβ2. Typically, this is achieved by the introduction of a heterologous nucleic acid into the cell to direct the expression of a polypeptide such as RARβ2. The polypeptide may be the same as the biological molecule or it may modulate the levels of the biological molecule as described above.

In general, simply modulating the levels of a biological molecule in one of two identical cells and then measuring gene transcription is not the aim of the methods of the present invention since you will be measuring the effect of the biological molecule on gene expression in the cells rather than using the change in the levels of the biological molecule to enhance or reduce the response to an event of interest.

However, where the biological molecule is a gene product, such as a polypeptide, that is produced naturally within the cell, altering the levels of the gene product by the introduction of a heterologous nucleic acid may be used to simultaneously both perturb a cellular process and enhance the response to such a perturbation making it easier to identify gene products involved in that cellular process using differential expression techniques. By way of an example, overexpression of HIF-1α not only induces an hypoxic response but amplifies the downstream elements of that response due an enhanced regulatory effect on HIF-1⊕ mediated transcription.

Nonetheless in the broader aspects of the present invention, two main possibilities arise. Firstly, the two cells are different and have inherently different gene expression patterns. In this situation, alterations in the levels of the biological molecule can be used to enhance those differences. The two cells may be, for example, from different tissues, or from different stages in development or differentiation. The two cells may also be different by virtue of one cell being from diseased tissue and the other cell from normal tissue. Other configurations envisaged are given above.

Secondly, the two cells are the same but one of the cells is stimulated in some manner and the other cell not (or one is stimulated to a greater extent than the other). For example, one cell is incubated in the presence of a growth factor and the other is not. The growth factor is therefore not the biological molecule but is instead a stimulus designed to perturb gene expression in the cell, the effects of which may be amplified by the biological molecule which in turn is altered in level by the polypeptide expressed from the heterologous nucleic acid.

Thus in a second aspect there is provided a method whereby genes whose expression is regulated by a signal are identified by subjecting two distinct cell populations to different levels of a signal, whereby either or both cells have been manipulated so as to alter the levels of a biological molecule whose activity is responsive to the signal, and identifying gene products whose expression differs. The term “whose activity is response to the signal” includes biological molecule whose concentration in the cell varies in response to the signal as well as biological molecules whose properties such as enzymatic activity or affinity for another cellular component varies in response to the signal.

Thus returning to our factor example, the cells that are exposed to the factor may have been altered to express increased levels of a transcription factor involved in the signal transduction cascade. Consequently, the effect of the growth factor will be increased downstream of the transcription factor (in either a negative or positive sense) making it easier to identify differentially expressed genes whose expression is regulated by the transcription factor and ultimately by the factor. Preferably the factor leads to stimulation of neural regeneration/neurite outgrowth via signalling through RARβ2.

The signal may be either physical, such as redox conditions, CO₂ levels, light or temperature, or chemical such as ligands that bind to receptors on the cell surface and trigger signal transduction pathways (including hormones or cell surface molecules normally attached to other cells), or substrates for enzyme reactions that diffuse into or are transported into the cell.

The first cell is subjected the signal at a first level and the second cell is subjected to the signal at a second level. The first level may simply be the absence of the signal and the second level may be the presence of the signal, or vice-versa. The levels of the signals may be adjusted so as to provide a discernible difference in gene expression but are preferably at physiologically relevant levels.

In another aspect of the present invention, knowledge already acquired about genes involved in a disease or other biological process may be used to generate further information about other genes whose expression is altered in a disease or other biological process. To do this, one cell is modified so that the levels of the gene product known to be involved in the disease or other biological process are altered, either directly by the introduction of a heterologous nucleic acid encoding the gene product, or indirectly as described above. Gene expression is then measured in both cells and the results compared to identify gene products whose expression varies.

In this aspect of the invention, the two cells may be identical, except for the change in the levels of the gene product known to be involved in the disease or other biological process of interest. The two cells may thus both be normal cells of the same type as a cell type in which the disease or other process manifests itself, or they may both be diseased cells. Alternatively, one cell may be normal and the other diseased. Preferably the diseased cell is the modified cell if only one of the cells is modified.

In another aspect of the invention, differential expression screening methods are used to identify genes involved in a disease or other process in a two stage procedure. Firstly, gene expression is compared between a first cell of interest, for example a cell from a normal patient, and a second cell of interest, for example corresponding cells from a diseased patient. As discussed above, the first cell and the second cell will be different in some aspect such that they have different expression patterns. This may be because the cells are from different tissues or different individuals (for example a normal patient and a diseased patient) or the cells may be of similar origin but have been treated differently in some respect.

Gene products whose expression differs between the first cell and the second cell are identified. Secondly, a third cell of interest, essentially identical to the first cell is used in a screening procedure where a candidate gene is introduced into the third cell so that levels of the genes are altered (typically raised). Gene expression in this cell is compared with gene expression in the first cell and gene products whose expression differs between the normal cell and the third cell comprising altered levels of the candidate gene are identified. If a gene product whose expression is altered in the second cell also has altered gene expression in the third cell, then the candidate gene is selected for further study. Preferably there is a correlation over two or more gene products, preferably at least four or five gene products to minimise false positives.

Clearly, the methods of the present invention may advantageously be applied to the differential analysis of non-dividing neuronal cells and a different sample of the same cells which have been induced to regenerate or undergo neurite outgrowth by the methods of the present invention. This differential analysis applies to the discovery and/or validation of candidate molecules, in particular those biological molecules which lie in the signalling pathway between the activation of the RARβ2 receptor and the actual morphological phenotype of neurite outgrowth. This phenomenon of neurite outgrowth/regeneration will be brought about by physiological changes within the cell which are initiated by the activation of RARβ2, and may include changes in gene expresison. Thus, by taking a sample of neuronal cells and introducing RARβ2 as described herein, and allowing retinoic acid to signal through this receptor, and comparing the pattern of gene expression with a sample of such cells which do not contain RARβ2/retinoic acid, key difference(s) in gene expression may be identified. The pathway(s) leading to neurite outgrowth will be switched on in the cells with RARβ2/retinoic acid. By making cDNA from these and from the non-activated cells in parallel, subtractive cDNA libraries may be made in order to isolate differences in gene expression between the two sets of cells. This or other differential screening technique(s), or proteomic techniques such as 2-D electrophoretic mapping, can be used to detect the stimulation and/or repression of particular gene(s) or sets of genes which the different conditions produce. These differentially expressed genes and/or their gene products are each individual candidate factors in the stimulation of neurite outgrowth, and it will be clearly understood that the invention relates also to these. This topic is discussed in more detail below.

Host Cells

Polynucleotides for use in the present invention—such as for use as targets or for expressing targets or for use as the pharmaceutically active agent—may be introduced into host cells.

The term “host cell”—in relation to the present invention includes any cell that could comprise the polynucleotide sequence of the present invention.

Here, polynucleotides may be introduced into prokaryotic cells or eukaryotic cells, for example yeast, insect or mammalian cells.

Polynucleotides of the invention may introduced into suitable host cells using a variety of techniques known in the art, such as transfection, transformation and electroporation. Where polynucleotides of the invention are to be administered to animals, several techniques are known in the art, for example infection with recombinant viral vectors such as retroviruses, herpes simplex viruses, adenoviruses, adeno-associated viruses, direct injection of nucleic acids and biolistic transformation. The selection of the particular technique for the administration of polynucleotides into particular host cell(s) is well within the abilities of a person skilled in the art and is further discussed herein. For example, a person wishing to administer polynucleotide to a non-dividing mammalian cell such as a neuronal cell would select a vector system capable or transfecting/transducing non-dividing mammalian cells. An example of such a vector is a viral vector such as a vector based on or derived from EIAV. This and further examples are discussed at length herein.

Thus, a further embodiment of the present invention provides host cells transformed or transfected with a polynucleotide that is or expresses the target of the present invention. Preferably said polynucleotide is carried in a vector for the replication and expression of polynucleotides that are to be the target or are to express the target. The cells will be chosen to be compatible with the said vector and may for example be prokaryotic (for example bacterial), fungal, yeast or plant cells.

The gram negative bacterium E. coli is widely used as a host for heterologous gene expression. However, large amounts of heterologous protein tend to accumulate inside the cell. Subsequent purification of the desired protein from the bulk of E. coli intracellular proteins can sometimes be difficult.

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide encoding the polypeptide of the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus licheniformis, Bacillus amyloliquefaciens, Kluyveromyces lactis and Saccharomyces cerevisiae.

Polypeptides that are extensively modified may require correct processing to complete their function. In those instances, mammalian cell expression systems (such as HEK-293, CHO, HeLA) are required, and the polypeptides are expressed either intracellularly, on the cell membranes, or secreted in the culture media if preceded by an appropriate leader sequence.

The use of suitable host cells—such as yeast, fungal, plant and mammalian host cells—may provide for post-translational modifications (e.g. myristoylation, glycosylation, truncation, lipidation and tyrosine, serine or threonine phosphorylation) as may be needed to confer optimal biological activity on recombinant expression products of the present invention.

Organism

The term “organism” in relation to the present invention includes any organism that could comprise the sequence according to the present invention and/or products obtained therefrom. Examples of organisms may include a fungus, yeast or a plant.

The term “transgenic organism” in relation to the present invention includes any organism that comprises the target according to the present invention and/or products obtained.

Transformation of Host Cells/Host Organisms

As indicated earlier, the host organism can be a prokaryotic or a eukaryotic organism. Examples of suitable prokaryotic hosts include E. coli and Bacillus subtilis. Teachings on the transformation of prokaryotic hosts is well documented in the art, for example see Sambrook et al (Molecular Cloning; A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

In another embodiment the transgenic organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence of the present invention into a construct designed for expression in yeast. Several types of constructs used for heterologous expression have been developed. The constructs contain a promoter active in yeast fused to the nucleotide sequence of the present invention, usually a promoter of yeast origin, such as the GAL1 promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces according to the present invention can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, eg G418.

Another host organism is a plant. The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

Further hosts suitable for the nucleotide sequence of the present invention include higher eukaryotic cells, such as insect cells or vertebrate cells, particularly mammalian cells, including human cells, or nucleated cells from other multicellular organisms. In recent years propagation of vertebrate cells in culture (tissue culture) has become a routine procedure. Examples of useful mammalian host cell lines are epithelial or fibroblastic cell lines such as Chinese hamster ovary (CHO) cells, NIH 3T3 cells, HeLa cells or 293T cells.

The nucleotide sequence of the present invention may be stably incorporated into host cells or may be transiently expressed using methods known in the art. By way of example, stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene to monitor transfection efficiency.

To produce such stably or transiently transfected cells, the cells should be transfected with a sufficient amount of the nucleotide sequence of the present invention. The precise amounts of the nucleotide sequence of the present invention may be empirically determined and optimised for a particular cell and assay.

Thus, the present invention also provides a method of transforming a host cell with a nucleotide sequence that is to be the target or is to express the target. Host cells transformed with the nucleotide sequence may be cultured under conditions suitable for the expression of the encoded protein. The protein produced by a recombinant cell may be displayed on the surface of the cell. If desired, and as will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain which will facilitate purification of soluble proteins (Kroll D J et al (1993) DNA Cell Biol 12:441-53).

Receptors

The RARβ2 receptor as discussed herein includes mimetics, homologues, fragments and part or all of the entire gene product. Preferably the RARβ2 receptor as discussed herein refers to substantially the entire gene product.

In one embodiment, the present invention relates to the use of a receptor in the production of neurite outgrowth. Previously, attempts have been made to produce neurite outgrowth using a number of different techniques. Typically, nerve growth factor (NGF) is used to stimulate neurite outgrowth. However, NGF is a relatively large molecule with a correspondingly high molecular weight, and is susceptible to protease mediated degradation. NGF is also relatively expensive to prepare. Similar approaches to the stimulation of neurite outgrowth have also encountered various difficulties. Moreover, such approaches have centred on the use of stimulatory factors such as growth factors in order to produce such desired phenotype(s). However, it is surprisingly shown herein that the long-felt need for the production of neurite outgrowth, for example in non-dividing cells, may be achieved using the converse approach disclosed herein, ie. the use of receptors to stimulate neurite outgrowth as described and demonstrated in the present invention. This disclosure runs against current thinking in the art, which has been focussed on the use of growth factors to try to elicit neurite outgrowth from non-dividing cells such as terminally differentiated neuronal cells. The surprising finding that receptor(s) may be delivered to such cells to produce neural regeneration/neurite outgrowth is illustrated herein by using RARβ2 as an example of this general approach. Thus, the present invention relates to the use of a receptor in the production of neurite outgrowth. The receptor may be any eukaryotic receptor, preferably a vertebrate receptor, more preferably a mammalian receptor, more preferably a primate receptor, most preferably a human receptor. Receptors for use in the present invention may comprise one or more membrane-spanning domain(s). In a preferred embodiment, receptors useful in the present invention are human receptors, without regard to their natural temporal and/or spatial expression profile. In a highly preferred embodiment, receptors useful in the present invention are human receptors which are not normally expressed in cell(s) of the adult target tissue. In a most highly preferred embodiment, receptors useful in the present invention are retinoic acid receptors such as RARs, such as in particular RARβ2. Receptor(s) useful in the present invention are preferably delivered to the target cell(s) using a vector system as described herein, such as a lentiviral vector system.

Neurological Disorders

Clearly, stimulation of neurite outgrowth according to the present invention will have therapeutic benefit in a number of pathologies. These include, but are not limited to, neurological disorders, for example degenerate neurological disorders such as Parkinson's disease, Alzheimer's syndrome, or related conditions, or neural injury such as spinal cord injury or other such physical condition.

The term neurological disorders as used herein may refer to any injury, whether mechanically (for example by trauma) or chemically induced (for example by neurotoxin(s), or by an regime of treatment having an immunosuppressant effect, whether by design, or as a side-effect), any neural pathology such as caused by viral infection or otherwise, any degenerative disorder, or other nerve tissue related disorder.

Examples of neurological disorders include conditions such as Parkinson's disease, Alzheimer's disease, senility, motor neurone disease, schizophrenia as well as other neural and/or neurodegenerative disorders. Other neural related disorders may include glaucoma or other cause of damage to the optic nerve, Bell's palsy or other forms of localised paralysis, neurally based impotence such as caused by nerve trauma following radical prostatectomy, or other complaints. Other disorders in which the invention may be useful include neuropathological effects of diabetes, AIDS neuropathy, leprosy etc.

The term neurological disorder refers to any disorder of a nervous system, whether the peripheral nervous system or the central nervous system (CNS), whether the sympathetic nervous system, or the parasympathetic nervous system, or whether affecting a subset or superset of different nerve types.

Nucleotide of Interest (NOI)

In accordance with the present invention, the NOI sequence may encode a peptide which peptide may be the pharmaceutically active agent—such as an RA receptor, preferably RARβ2, or an agonist thereof.

Such coding NOI sequences may be typically operatively linked to a suitable promoter capable of driving expression of the peptide, such as in one or more specific cell types.

In addition to the NOI or part thereof and the expression regulatory elements described herein, the delivery system may contain additional genetic elements for the efficient or regulated expression of the gene or genes, including promoters/enhancers, translation initiation signals, internal ribosome entry sites (IRES), splicing and polyadenylation signals.

The NOI or NOIs may be under the expression control of an expression regulatory element, usually a promoter or a promoter and enhancer. The enhancer and/or promoter may be preferentially active in neural cells, such that the NOI is preferentially expressed in the particular cells of interest, such as in nerve cells. Thus any significant biological effect or deleterious effect of the NOI on the individual being treated may be reduced or eliminated. The enhancer element or other elements conferring regulated expression may be present in multiple copies. Likewise, or in addition, the enhancer and/or promoter may be preferentially active in one or more specific cell types—such as neural cells for example post-mitotically terminally differentiated non-replicating cells such as neurons.

The term “promoter” is used in the normal sense of the art, e.g. an RNA polymerase binding site in the Jacob-Monod theory of gene expression.

The term “enhancer” includes a DNA sequence which binds to other protein components of the transcription initiation complex and thus facilitates the initiation of transcription directed by its associated promoter.

Expression Vector

Preferably, the NOI (e.g. that encoding RARβ2 or part thereof used in the method of the present invention is inserted into a vector which is operably linked to a control sequence that is capable of providing for the expression of the coding sequence by the host cell, i.e. the vector is an expression vector.

Codon Optimisation

As used herein, the terms “codon optimised” and “codon optimisation” refer to an improvement in codon usage. By way of example, alterations to the coding sequences for viral components may improve the sequences for codon usage in the mammalian cells or other cells which are to act as the producer cells for retroviral vector particle production. This is referred to as “codon optimisation”. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Preferably a high titre lentiviral vector is produced using a codon optimised gag and a codon optimised pol or a codon optimised env (see seq. listing and/or WO99/41397).

Preferably a high titre retroviral vector is produced using a modified and/or extended packaging signal.

Packaging Signal

As used herein, the term “packaging signal” or “packaging sequence” refers to sequences located within the retroviral genome which are required for insertion of the viral RNA into the viral capsid or particle. Several retroviral vectors use the minimal packaging signal (also referred to as the psi sequence) needed for encapsidation of the viral genome. By way of example, this minimal packaging signal encompasses bases 212 to 563 of the Mo-MLV genome (Mann et al 1983: Cell 33: 153). As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Preferably a high titre lentiviral vector is produced using a modified packaging signal.

Preferably the lentiviral construct is a based on an EIAV vector genome where all the accessory genes are removed except Rev.

Accessory Genes

As used herein, the term “accessory genes” refer to a variety of virally encoded accessory proteins capable of modulating various aspects of retroviral replication and infectivity. These proteins are discussed in Coffin et al (ibid) (Chapters 6 and 7). Examples of accessory proteins in lentiviral vectors include but are not limited to tat, rev, nef, vpr, vpu, vif, vpx. An example of a lentiviral vector useful in the present invention is one which has all of the accessory genes removed except rev.

Transcriptional Control

The control of proviral transcription remains largely with the noncoding sequences of the viral LTR. The site of transcription initiation is at the boundary between U3 and R in the left hand side LTR and the site of poly (A) addition (termination) is at the boundary between R and U5 in the right hand side LTR. The 3′U3 sequence contains most of the transcriptional control elements of the provirus, which include the promoter and multiple enhancer sequences responsive to cellular and in some cases, viral transcriptional activator proteins.

An LTR present, for example, in a construct of the present invention and as a 3′LTR in the provirus of, for example, a target cell of the invention may be a native LTR or a heterologous regulatable LTR. It may also be a transcriptionally quiescent LTR for use in SIN vector technology.

The term “regulated LTR” also includes an inactive LTR such that the resulting provirus in the target cell can not produce a packagable viral genome (self-inactivating (SIN) vector technology).

Preferably the regulated retroviral vector of the present invention is a self-inactivating (SIN) vector.

Self-Inactivating (SIN) Vector

By way of example, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al 1986 Proc Natl Acad Sci 83: 3194-3198; Dougherty and Temin 1987 Proc Natl Acad Sci 84: 1197-1201; Hawley et al 1987 Proc Natl Acad Sci 84: 2406-2410; Yee et al 1987 Proc Natl Acad Sci 91: 9564-9568). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolly et al 1983 Nucleic Acids Res 11: 1855-1872) or suppression of transcription (Emerman and Temin 1984 Cell 39: 449467). This strategy can also be used to eliminate downstream transcription from the 3′ LTR into genomic DNA (Herman and Coffin 1987 Science 236: 845-848). This is of particular concern in human gene therapy where it is of critical importance to prevent the adventitious activation of an endogenous oncogene.

Targeted Vector

The term “targeted vector” refers to a vector whose ability to infect/transfect/transduce a cell or to be expressed in a host and/or target cell is restricted to certain cell types within the host organism, usually cells having a common or similar phenotype.

Preferably the targeted vector has a pseudotyped envelope gene in order to effectively transduce a specific cell type.

Envelope (ENV)

If the retroviral component includes an env nucleotide sequence, then all or part of that sequence can be optionally replaced with all or part of another env nucleotide sequence such as, by way of example, the amphotropic Env protein designated 4070A or the influenza haemagglutinin (HA) or the vesicular stomatitis virus G (VSV-G) protein. Replacement of the env gene with a heterologous env gene is an example of a technique or strategy called pseudotyping. Examples of pseudotyping may be found in WO-A-98/05759, WO-A-98/05754, WO-A-97/17457, WO-A-96/09400, WO-A-91/00047 and Mebatsion et al 1997 Cell 90, 841-847.

In one preferred aspect, the retroviral vector of the present invention has been pseudotyped. In this regard, pseudotyping can confer one or more advantages. For example, with the lentiviral vectors, the env gene product of the HIV based vectors would restrict these vectors to infecting only cells that express a protein called CD4. But if the env gene in these vectors has been substituted with env sequences from other RNA viruses, then they may have a broader infectious spectrum (Verma and Somia 1997 Nature 389:239-242). By way of example, workers have pseudotyped an HIV based vector with the glycoprotein from VSV (Verma and Somia 1997 ibid).

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al 1996 Gene Therapy 3: 280-6; Fielding et al 1998 Blood 9: 1802 and references cited therein).

The term “retroviral vector particle” refers to the packaged retroviral vector, that is preferably capable of binding to and entering target cells. The components of the particle, as already discussed for the vector, may be modified with respect to the wild type retrovirus. For example, the Env proteins in the proteinaceous coat of the particle may be genetically modified in order to alter their targeting specificity or achieve some other desired function.

Preferably, the viral vector preferentially transduces a certain cell type or cell types.

More preferably, the viral vector is a targeted vector, that is it has a tissue tropism which is altered compared to the native virus, so that the vector is targeted to particular cells.

For retroviral vectors, this may be achieved by modifying the Env protein. The Env protein of the retroviral secondary vector needs to be a non-toxic envelope or an envelope which may be produced in non-toxic amounts within the primary target cell, such as for example a MMLV amphotropic envelope or a modified amphotropic envelope. The safety feature in such a case is preferably the deletion of regions or sequence homology between retroviral components.

Preferably the envelope is one which allows transduction of human cells. Examples of suitable env genes include, but are not limited to, VSV-G, a MLV amphotropic env such as the 4070A env, the RD114 feline leukaemia virus env or haemagglutinin (HA) from an influenza virus. The Env protein may be one which is capable of binding to a receptor on a limited number of human cell types and may be an engineered envelope containing targeting moieties. The env and gag-pol coding sequences are transcribed from a promoter and optionally an enhancer active in the chosen packaging cell line and the transcription unit is terminated by a polyadenylation signal. For example, if the packaging cell is a human cell, a suitable promoter-enhancer combination is that from the human cytomegalovirus major immediate early (hCMV-MIE) gene and a polyadenylation signal from SV40 virus may be used. Other suitable promoters and polyadenylation signals are known in the art.

The packaging cell may be an in vivo packaging cell in the body of an individual to be treated or it may be a cell cultured in vitro such as a tissue culture cell line. Suitable cell lines include mammalian cells such as murine fibroblast derived cell lines or human cell lines. Preferably the packaging cell line is a human cell line, such as for example: 293 cell line, HEK293, 293-T, TE671, HT1080.

Alternatively, the packaging cell may be a cell derived from the individual to be treated such as a monocyte, macrophage, stem cells, blood cell or fibroblast. The cell may be isolated from an individual and the packaging and vector components administered ex vivo followed by re-administration of the autologous packaging cells. Alternatively the packaging and vector components may be administered to the packaging cell in vivo. Methods for introducing retroviral packaging and vector components into cells of an individual are known in the art. For example, one approach is to introduce the different DNA sequences that are required to produce a retroviral vector particle e.g. the env coding sequence, the gag-pol coding sequence and the defective retroviral genome into the cell simultaneously by transient triple transfection (Landau & Littman 1992 J. Virol. 66, 5110; Soneoka et al 1995 Nucleic Acids Res 23:628-633).

In one embodiment the vector configurations of the present invention use as their production system, three transcription units expressing a genome, the gag-pol components and an envelope. The envelope expression cassette may include one of a number of envelopes such as VSV-G or various murine retrovirus envelopes such as 4070A.

Conventionally these three cassettes would be expressed from three plasmids transiently transfected into an appropriate cell line such as 293T or from integrated copies in a stable producer cell line. An alternative approach is to use another virus as an expression system for the three cassettes, for example baculovirus or adenovirus. These are both nuclear expression systems. To date the use of a poxvirus to express all of the components of a retroviral or lentiviral vector system has not been described. In particular, given the unusual codon usage of lentiviruses and their requirement for RNA handling systems such as the rev/RRE system it has not been clear whether incorporation of all three cassettes and their subsequent expression in a vector that expresses in the cytoplasm rather than the nucleus is feasible. Until now the possibility remained that key nuclear factors and nuclear RNA handling pathways would be required for expression of the vector components and their function in the gene delivery vehicle. Here we describe such a system and show that lentiviral components can be made in the cytoplasm and that they assemble into functional gene delivery systems. The advantage of this system is the ease with which poxviruses can be handled, the high expression levels and the ability to retain introns in the vector genomes.

According to another aspect therefore there is provided a hybrid viral vector system for in vivo gene delivery, which system comprises a primary viral vector which is obtainable from or is based on a poxvirus and a second viral vector which is obtainable from or is based on a retroviral vector, preferably a lentiviral vector, even more preferably a non-primate lentiviral vector, even more preferably an EIAV.

The secondary vector may be produced from expression of essential genes for retroviral vector production encoded in the DNA of the primary vector. Such genes may include a gag-pol from a retrovirus, an env gene from an enveloped virus and a defective retroviral vector containing one or more therapeutic or diagnostic NOI(s). The defective retroviral vector contains in general terms sequences to enable reverse transcription, at least part of a 5′ long terminal repeat (LTR), at least part of a 3′LTR and a packaging signal.

If it is desired to render the secondary vector replication defective, that secondary vector may be encoded by a plurality of transcription units, which may be located in a single or in two or more adenoviral or other primary vectors.

In some therapeutic or experimental situations it may be desirable to obviate the need to make EIAV derived from MVA in vitro. MVA-EIAV hybrids are delivered directly into the patient/animal e.g. MVA-EIAV is injected intravenously into the tail vein of a mouse and this recombinant virus infects a variety of murine tissues e.g. lung, spleen etc. Infected cells express transduction competent EIAV containing a therapeutic gene for gene therapy for example. EIAV vector particles bud from these cells and transduce neighbouring cells. The transduced cell then contains an integrated copy of the EIAV vector genome and expresses the therapeutic gene product or other gene product of interest. If expression of the therapeutic gene product is potentially toxic to the host it may be regulated by a specific promoter, e.g. the hypoxic response element (HRE), which will restrict expression to those cells in a hypoxic environment. For gene therapy of lung/trachea epithelium cells e.g to treat cystic fibrosis MVA-EIAV may be given as an aerosol delivered intranasally. Alternatively, macrophages can be transduced in vitro and then reintroduced to create macrophage factories for EIAV-based vectors. Furthermore, because MVA is replication incompetent MVA-EIAV hybrids could also be used to treat immuno-suppressed hosts.

Vaccinia virus, the prototypic member of the orthopox genus within the family poxviridae, was the first virus used for expression of recombinant exogenous proteins (Mackett et al 1982, Paoletti & Panicalli 1982). Vaccinia virus has a large DNA genome of greater than 180 kb and reports indicate that it can accommodate over 25 kb of foreign DNA (Merchlinsky & Moss 1992). Several other strains of poxviruses have been adapted as recombinant expression vectors (for review see Carroll and Moss 1997) e.g. fowlpox (Taylor & Paoletti 1988), canarypox (Taylor et al 1991), swinepox (van der Leek et al 1994) and entomopox (Li et al 1997). Additionally, due to safety concerns, several highly attenuated strains of vaccinia virus have been developed that are compromised in human and other mammalian cells e.g. modified vaccinia virus Ankara (MVA) (Mayr 1978, Sufter 1992), NYVAC (Paolefti et al 1994), vaccinia virus deficient in a DNA replication enzyme (Holzer et al 1997). These may all be used in the present invention.

MVA was derived from a replication competent vaccinia smallpox vaccine strain, Ankara. After >500 passages in chick embryo fibroblast cells the virus isolate was shown to be highly attenuated in a number of animal models including mice that were immune deficient (Mayr et al 1978). The attenuated isolate, MVA, was used to vaccinate over 120,000 people, many of which were immunocompromised (Mahnel 1994) without adverse effects. Studies illustrate that MVA can infect a wide range of mammalian cells but productive infection has only been observed in Hamster kidney cell BHK-21 (Carroll 1997). In all other tested mammalian cell lines early expression, DNA replication and late expression are observed leading to the production of non-infectious immature virus particles (Carroll 1997, Meyer 1991). Virus replication studies show that a minority of mammalian cells can support very low level production of infectious virus i.e. BS-C-1 cells in which 1 infectious MVA particle is produced per cell (Carroll and Moss 1997). Late gene expression usually give rise to >10 fold more protein that those genes under early promoters (Chakrabarti et al 1997, Wyatt et al 1996). In all other attenuated poxvirus strains late gene expression is rarely observed in mammalian cells.

Production of retrovirus vector systems e.g. MLV-HIV and lentivirus vector systems requires the construction of producer lines that express the virus genome and essential structural proteins to make transduction competent virus. Generally, this is a relatively inefficient process which is further complicated when the virus is pseudotyped with toxic envelope proteins such as VSV-G. Expression of a functional genome and the required structural proteins from within a recombinant poxvirus may obviate many of the current inefficient retrovirus and lentivirus vector production technologies. Additionally, such recombinant poxviruses may be directly injected into patients to give rise to in vivo production of retrovirus or lentivirus.

MVA is a particularly suitable poxvirus for the construction of a pox-retrovirus or pox-lentivirus hybrid due to its non-replicating phenotype and its ability to perform both early and strong late expression for the production of high titre vector preparations.

Replication Vectors

The nucleotide sequences encoding the of the present invention may be incorporated into a recombinant replicable vector. The vector may be used to replicate the nucleotide sequence in a compatible host cell. Thus in one embodiment of the present invention, the invention provides a method of making the RARβ2 of the present invention by introducing a nucleotide sequence of the present invention into a replicable vector, introducing the vector into a compatible host cell, and growing the host cell under conditions which bring about replication of the vector. The vector may be recovered from the host cell.

Host/Target Cells

Host and/or target cells comprising nucleotide sequences of the present invention may be used to express the RARβ2 of the present invention under in vitro, in vivo and ex vivo conditions.

The term “host cell” and/or “target cell” includes any cell derivable from a suitable organism which a vector is capable of transfecting or transducing. Examples of host and/or target cells can include but are not limited to cells capable of expressing the RARβ2 of the present invention under in vitro, in vivo and ex vivo conditions. Examples of such cells include but are not limited to neuronal cells, nerve cells, post-mitotically terminally differentiated non-replicating cells such as neurons or combinations thereof.

In a preferred embodiment, the cell is a mammalian cell.

In a highly preferred embodiment, the cell is a human cell.

The term “organism” includes any suitable organism. In a preferred embodiment, the organism is a mammal. In a highly preferred embodiment, the organism is a human.

The present invention also provides a method comprising transforming a host and/or target cell with a or the nucleotide sequence(s) of the present invention.

The term “transformed cell” means a host cell and/or a target cell having a modified genetic structure. With the present invention, a cell has a modified genetic structure when a vector according to the present invention has been introduced into the cell.

Regulation of Expression in vitro/vivo/ex vivo

The present invention also encompasses gene therapy whereby the RARβ2 encoding nucleotide sequence(s) of the present invention is regulated in vitrolin vivolex vivo. For example, expression regulation may be accomplished by administering compounds that bind to the RARβ2 encoding nucleotide sequence(s) of the present invention, or control regions associated with the RARβ2 encoding nucleotide sequence of the present invention, or its corresponding RNA transcript to modify the rate of transcription or translation.

Control Sequences

Control sequences operably linked to sequences encoding the RARβ2 of the present invention include promoters/enhancers and other expression regulation signals. These control sequences may be selected to be compatible with the host cell and/or target cell in which the expression vector is designed to be used. The control sequences may be modified, for example by the addition of further transcriptional regulatory elements to make the level of transcription directed by the control sequences more responsive to transcriptional modulators.

Operably Linked

The term “operably linked” means that the components described are in a relationship permitting them to function in their intended manner. A regulatory sequence “operably linked” to a coding sequence is ligated in such a way that expression of the coding sequence is achieved under condition compatible with the control sequences.

Preferably the nucleotide sequence of the present invention is operably linked to a transcription unit.

The term “transcription unit(s)” as described herein are regions of nucleic acid containing coding sequences and the signals for achieving expression of those coding sequences independently of any other coding sequences. Thus, each transcription unit generally comprises at least a promoter, an optional enhancer and a polyadenylation signal.

Promoters

The term promoter is well-known in the art and is used in the normal sense of the art, e.g. an RNA polymerase binding site. The term encompasses nucleic acid regions ranging in size and complexity from minimal promoters to promoters including upstream elements and enhancers.

The promoter is typically selected from promoters which are functional in mammalian, cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase).

Preferably the promoter is a constitutive promoter such as CMV.

Preferably the promoters of the present invention are tissue specific.

Tissue-Specific Promoters

The promoters of the present invention may be tissue-specific promoters. Examples of suitable tissue restricted promoters/enhancers are those which are highly active in tumour cells such as a promoter/enhancer from a MUC1 gene, a CEA gene or a 5T4 antigen gene. Examples of temporally restricted promoters/enhancers are those which are responsive to ischaemia and/or hypoxia, such as hypoxia response elements or the promoter/enhancer of a grp78 or a grp94 gene. The alpha fetoprotein (AFP) promoter is also a tumour-specific promoter. One preferred promoter-enhancer combination is a human cytomegalovirus (hCMV) major immediate early (MIE) promoter/enhancer combination.

Preferably the promoters of the present invention are tissue specific. That is, they are capable of driving transcription of a RARβ2 encoding nucleotide sequence(s) in one tissue while remaining largely “silent” in other tissue types.

The term “tissue specific” means a promoter which is not restricted in activity to a single tissue type but which nevertheless shows selectivity in that they may be active in one group of tissues and less active or silent in another group.

The level of expression of a or the RARβ2 encoding nucleotide sequence(s) under the control of a particular promoter may be modulated by manipulating the promoter region. For example, different domains within a promoter region may possess different gene regulatory activities. The roles of these different regions are typically assessed using vector constructs having different variants of the promoter with specific regions deleted (that is, deletion analysis). This approach may be used to identify, for example, the smallest region capable of conferring tissue specificity.

A number of tissue specific promoters, described above, may be particularly advantageous in practising the present invention. In most instances, these promoters may be isolated as convenient restriction digestion fragments suitable for cloning in a selected vector. Alternatively, promoter fragments may be isolated using the polymerase chain reaction. Cloning of the amplified fragments may be facilitated by incorporating restriction sites at the 5′ end of the primers. Preferably, a tissue-specific promoter used herein is specific for neuronal cells.

Inducible Promoters

The promoters of the present invention may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

Enhancer

In addition, any of these promoters may be modified by the addition of further regulatory sequences, for example enhancer sequences. Chimeric promoters may also be used comprising sequence elements from two or more different promoters described above.

The term “enhancer” includes a DNA sequence which binds to other protein components of the transcription initiation complex and thus facilitates the initiation of transcription directed by its associated promoter.

The in vitro/in vivolex vivo expression of the RARβ2 of the present invention may be used in combination with a protein of interest (POI) or a nucleotide sequence of interest (NOI) encoding same.

POIs and NOIs

Suitable proteins of interest (POIs) or NOIs encoding same for use in the present invention include those that are of therapeutic and/or diagnostic application such as, but are not limited to: sequences encoding cytokines, chemokines, hormones, antibodies, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppressor protein and growth factors, membrane proteins, vasoactive proteins and peptides, anti-viral proteins and ribozymes, and derivatives therof (such as with an associated reporter group). When included, the POIs or NOIs encoding same may be typically operatively linked to a suitable promoter, which may be a promoter driving expression of a ribozyme(s), or a different promoter or promoters, such as in one or more specific cell types.

Cytokines

In one aspect of the present invention the NOI(s) encodes a POI(s) wherein the POI is a cytokine or a cytokine receptor.

As used herein, the term “cytokines” refers to any varied group of proteins that are released from mammalian cells and act on other cells through specific receptors, said term also including said receptors. The term “cytokine” is often used interchangeably with the term “mediator”. Cytokines may elicit from the target cell a variety of responses depending on the cytokine and the target cell. By way of example, cytokines may be important in signalling between cells as inflammatory reactions develop. In the initial stages, cytokines such as IL-1 and IL-6 may be released from cells of the tissue where the inflammatory reaction is occurring. Once lymphocytes and mononuclear cells have started to enter the inflammatory site, they may become activated by antigen and release cytokines of their own such as IL-1, TNF, IL4 and IFNγ which further enhance cellular migration by their actions on the local endothelium. Other cytokines, such as IL-8, are chemotactic or can activate incoming cells. The term “cytokine” includes but is not limited to factors such as cardiotrophin, EGF, FGF-acidic, FGF-basic, flt3 Ligand, G-CSF, GM-CSF, IFN-γ, IGF-I, IGF-III, IL-1α, IL-1β, IL-2, IL-3, IL-4, IL-5, IL-6, IL-7, IL-8, IL-9, IL-10, IL-11, IL-12, IL-13, IL-15, IL-16, IL-17, IL-18 (IGIF), KGF, LIF, M-CSF, Oncostatin M, PDGF-A, PDGF-AB, PDGF-BB, SCF, SCGF, TGF-α, TGF-β₁, TNF-α, TNF-β, TPO and VEGF, as well as their cognate receptors.

Coupling

The RARβ2 of the present invention can be coupled to other molecules using standard methods. The amino and carboxyl termini of RARβ2 may be isotopically and nonisotopically labeled with many techniques, for example radiolabeling using conventional techniques (tyrosine residues—chloramine T, iodogen, lactoperoxidase; lysine residues—Bolton-Hunter reagent). These coupling techniques are well known to those skilled in the art. The coupling technique is chosen on the basis of the functional groups available on the amino acids including, but not limited to amino, sulfhydral, carboxyl, amide, phenol, and imidazole. Various reagents used to effect these couplings include among others, glutaraldehyde, diazotized benzidine, carbodiimide, and p-benzoquinone.

Chemical Coupling

The RARβ2 of the present invention may be chemically coupled to isotopes, enzymes, carrier proteins, cytotoxic agents, fluorescent molecules, radioactive nucleotides and other compounds for a variety of applications including but not limited to imaging/prognosis, diagnosis and/or therapy.

Imaging

The use of labelled RARβ2 of the present invention with short lived isotopes enables visualization quantitation of RARβ2 binding sites in vivo using autoradiographic, or modern radiographic or other membrane binding techniques such as positron emission tomography in order to locate tumours with RARβ2 binding sites. This application provides important diagnostic and/or prognostic research tools.

Conjugates

In other embodiments, the RARβ2 of the invention is coupled to a scintigraphic radiolabel, a cytotoxic compound or radioisotope, an RARβ2 for converting a non-toxic prodrug into a cytotoxic drug, a compound for activating the immune system in order to target the resulting conjugate to a disease site such as a colon tumour, or a cell-stimulating compound. Such conjugates have a “binding portion”, which consists of the RARβ2 of the invention, and a “functional portion”, which consists of the radiolabel,

Individual

As used herein, the term “individual” refers to vertebrates, particularly members of the mammalian species, more in particular, humans.

Treatment

It is to be appreciated that all references herein to treatment include curative, palliative and prophylactic treatment.

Dosage

The dosage of the RARβ2 and/or pharmaceutical composition of the present invention will depend on the disease state or condition being treated and other clinical factors such as weight and condition of the individual and the route of administration of the compound. Depending upon the half-life of the RARβ2 in the particular individual, the RARβ2 and/or pharmaceutical composition can be administered between several times per day to once a week. It is to be understood that the present invention has application for both human and veterinary use. The methods of the present invention contemplate single as well as multiple administrations, given either simultaneously or over an extended period of time.

Typically, a physician will determine the actual dosage which will be most suitable for an individual subject and it will vary with the age, weight and response of the particular patient and severity of the condition. The dosages below are exemplary of the average case. There can, of course, be individual instances where higher or lower dosage ranges are merited.

In addition or in the alternative the compositions (or component parts thereof) of the present invention may be administered by direct injection. In addition or in the alternative the compositions (or component parts thereof) of the present invention may be administered topically. In addition or in the alternative the compositions (or component parts thereof) of the present invention may be administered by inhalation. In addition or in the alternative the compositions (or component parts thereof) of the present invention may also be administered by one or more of: a mucosal route, for example, as a nasal spray or aerosol for inhalation or as an ingestable solution such as by an oral route, or by a parenteral route where delivery is by an injectable form, such as, for example, by a rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) transdermal, intraperitoneal, intracranial, intracerebroventricular, intracerebral, intravaginal, intrauterine, or parenteral (e.g., intravenous, intraspinal, intracavernosal, subcutaneous, transdermal or intramuscular) route.

By way of further example, the pharmaceutical composition of the present invention may be administered in accordance with a regimen of 1 to 10 times per day, such as once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Disorders

The present invention is believed to have a wide therapeutic applicability.

For example, the present invention may be useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of bums, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as comea, bone marrow, organs, lenses, pacemakers, natural or artificial skin tissue.

In particular, the present invention may be useful in the treatment of neurological disorders or injuries as discussed herein.

Delivery

The delivery system for use in the present invention may be any suitable delivery system for delivering said NOI and providing said NOI is expressed in vivo to produce said associated peptide (e.g. RARβ2), which in turn provides the beneficial therapeutic effect.

The delivery system may be a viral delivery system. Viral delivery systems include but are not limited to adenovirus vector, an adeno-associated viral (AAV) vector, a herpes viral vector, retroviral vector, lentiviral vector, baculoviral vector. Alternatively, the delivery system may be a non-viral delivery system—such as by way of example DNA transfection methods of, for example, plasmids, chromosomes or artificial chromosomes. Here transfection includes a process using a non-viral vector to deliver a gene to a target mammalian cell. Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), and combinations thereof.

Other examples of vectors include ex vivo delivery systems—which include but are not limted to DNA transfection methods such as electroporation, DNA biolistics, lipid-mediated transfection, compacted DNA-mediated transfection).

In a preferred aspect, the delivery system is a vector.

In a more preferred aspect, the delivery system is a viral delivery system—sometimes referred to as a viral vector.

Vectors

As it is well known in the art, a vector is a tool that allows or faciliates the transfer of an entity from one environment to another. By way of example, some vectors used in recombinant DNA techniques allow entities, such as a segment of DNA (such as a heterologous DNA segment, such as a heterologous cDNA segment), to be transferred into a target cell. Optionally, once within the target cell, the vector may then serve to maintain the heterologous DNA within the cell or may act as a unit of DNA replication. Examples of vectors used in recombinant DNA techniques include plasmids, chromosomes, artificial chromosomes or viruses.

The term “vector” includes expression vectors and/or transformation vectors.

The term “expression vector” means a construct capable of in vivo or in vitro/ex vivo expression.

The term “transformation vector” means a construct capable of being transferred from one species to another.

Viral Vectors

In the present invention, the NOI may be introduced into suitable host cells using a viral delivery system (a viral vector). A variety of viral techniques are known in the art, such as for example infection with recombinant viral vectors such as DNA viruses, retroviruses, herpes simplex viruses, adenoviruses and adeno-associated viruses.

Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al 1999 Human Gene Ther 10(10):1619-32). In the case of viral vectors, gene delivery is typically mediated by viral infection of a target cell.

Herpes Virus Based Vectors

Herpes simplex viruses (HSV) I and II are large linear DNA viruses of approximately 150 kb encoding 70-80 genes. Like adenoviruses, HSV can infect a wide variety of cell types, including muscle, tumours, lung, liver and pancreatic islets. The viruses are able both to infect cells lytically and to establish latency in specific cell types, such as neurons. In order to use HSV as a vector, it is rendered replication defective. Following infection of a cell with HSV, the expression of a small number of immediate early (IE) genes is induced by a viral transactivating protein, VP16, which is carried into the cell as part of the viral tegument. The IE genes, which include ICPO, 4, 6, 22 and 27, are themselves regulators of gene expression that are important for the induction of the early and late genes required for viral replication and encapsidation. Mutation of ICP4 results in a virus unable to replicate except in a complementing cell line, but which still expresses the other IE gene products; these other IE proteins are toxic to many cell types. Vectors defective for ICP4, 22 and 27 have been generated that have reduced levels of toxicity and prolonged gene expression in culture and in vivo. Herpes simplex virus can infect non-dividing cells of the mammalian nervous system.

An alternative approach to producing infectious HSV vectors is the use of amplicons. In this approach, a plasmid containing an HSV origin of replication and packaging sequence is cotransfected with cosmids containing the HSV genome but with a defective packaging sequence. The resulting virus particles contain only plasmid nucleic acid sequences, thereby eliminating any toxicity associated with low-level HSV-protein expression. This approach generates a helper free stock of virus. HSV vectors have a large capacity for inserting heterologous DNA, allowing up to 50 kb to be included successfully, which may comprise multiple therapeutic genes. For example, four different antitumour genes have been inserted into a single HSV vector for use in cancer therapy. HSV vectors can be used to obtain highly regulated gene expression. An RU486-hormone-regulated chimeric transcription factor has been inserted into HSV along with a promoter containing binding sites for the regulated transcription factor; specific, regulated gene expression has been observed in vivo. Essentially all of the viral proteins may be deleted (gut-less vectors), still allowing around 10⁶ viral particles to be produced per ml.

Adeno-Associated Viral Vectors

Adeno-associated virus (MV) is a member of the parvovirus family, small single-stranded DNA viruses that require a helper virus, such as adenovirus or herpes-simplex virus, for replication. MV is a human virus, with the majority of the population being seropositive for MV, but no pathology has been associated with it. The virus contains two genes, rep and cap, encoding polypeptides important for replication and encapsidation, respectively. The wild-type virus can be grown to high titres and is able to integrate stably into a specific region of chromosome 19 following infection. The recombinant virus may not always integrate site-specifically. It has been suggested that this integration requires the presence of the rep protein. In wild-type virus infection, second-strand synthesis is stimulated by the presence of adenovirus E1 and E4 proteins; in the absence of adenovirus coinfection, cellular factors appear to dictate the rate of second-strand synthesis. In certain cell types, and/or following treatment with DNA-damaging agents, the rate of second-strand synthesis is high.

For the production of viral vectors, these two genes can be supplied in trans with only the inverted terminal repeats (ITRs) required in cis for viral replication. Therapeutic genes with the appropriate regulatory sequences can be inserted between the two ITRs, and the viral vector generated by cotransfection into the 293 cell line with a rep and cap expression vector and subsequent infection with a first-generation adeno-viral vector.

The degree of MV infection of muscle, brain and liver cells with recombinant virus is exceedingly high in vivo. In these cell types, stable infection and gene expression apparently occurs independently of the helper virus. Injection of a β-galactosidase containing MV vector into muscle also has resulted in β-galactosidase-positive myofibres for up to two years. Similarly, the injection of virus into the brain has resulted in long-term gene expression. AAV vectors containing human factor IX complementary DNA have been used to infect liver and muscle cells in immunocompetent mice. The mice produced therapeutic amounts of factor IX protein in their blood for over six months, confirming the utility of MV as a viral vector. MV is highly suitable for the delivery of genes to specific target cells in vivo, preferably without inducing an immune response to the infected cells.

Retroviral Vectors

Examples of retroviruses include but are not limited to: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al “Retroviruses” 1997 Cold Spring Harbour Laboratory Press Eds: J M Coffin, S M Hughes, H E Varmus pp 758-763).

Preferred vectors for use in accordance with the present invention are recombinant viral vectors, in particular recombinant retroviral vectors (RRV) such as lentiviral vectors. Lentiviral vectors are able to deliver genes to non-dividing, terminally differentiated cells.

The term “recombinant retroviral vector” (RRV) refers to a vector with sufficient retroviral genetic information to allow packaging of an RNA genome, in the presence of packaging components, into a viral particle capable of infecting a target cell. Infection of the target cell includes reverse transcription and integration into the target cell genome. The RRV carries non-viral coding sequences which are to be delivered by the vector to the target cell. An RRV is incapable of independent replication to produce infectious retroviral particles within the final target cell. Usually the RRV lacks a functional gag-pol and/or env gene and/or other genes essential for replication.

Lentiviral genomes can be quite variable. For example there are many quasi-species of HIV-1 which are still functional. This is also the case for EIAV. These variants may be used to enhance particular parts of the transduction process. Examples of HIV-1 variants may be found at http://hiv-web.lanl.gov. Details of EIAV clones may be found at the NCBI database: http://www.ncbi.nim.nih.qov.

EIAV vectors have been shown to deliver genes very efficiently to a number of neuronal cell types in vitro and in vivo. Gene expression has been sustained for a number of months in vivo, with little or no immunological reaction. Thus, according to the present invention EIAV vectors are a suitable delivery system to direct expression of RARβ2 in the human peripheral and central nervous systems and such systems are discussed in detail herein.

Vector titre may be estimated by infection assays. For example, infections could be carried out with vector preparation in question, and antibody staining for the product of the nucleotide of interest could be used to determine the proportion of productively infected cells, giving an indication of the titre of the vector preparation. For example, antibodies directed against RARβ2 are commercially available and may be advantageously utilised for this purpose according to the manufacturers' instructions. Alternatively, a PCR approach may be used, by amplifying using primers directed at the nucleotide of interest delivered by the vector, such as a nucleotide sequence directing the expression of RARβ2. Primers may advantageously be designed to include or comprise vector sequence(s) in order to ensure that the relevant amplification product has indeed originated from the sequence in question. Other ways in which vector titre may be estimated are known in the art, and are discussed in the Examples section hereinbelow.

Non-Viral Delivery

The pharmaceutically active agent (e.g. the RARβ2) may be administered using non-viral techniques.

By way of example, the pharmaceutically active agent may be delivered using peptide delivery. Peptide delivery uses domains or sequences from proteins capable of translocation through the plasma and/or nuclear membrane

Polypeptides of interest such as RARβ2 may be directly introduced to the cell by microinjection, or delivery using vesicles such as liposomes which are capable of fusing with the cell membrane. Viral fusogenic peptides may also be used to promote membrane fusion and delivery to the cytoplasm of the cell.

Preferably, the RARβ2 or fragment(s) thereof may be delivered into cells as protein fusions or conjugates with a protein capable of crossing the plasma membrane and/or the nuclear membrane. Preferably, the RARβ2 or fragment(s) thereof is fused or conjugated to a domain or sequence from such a protein responsible for the translocational activity. Preferred translocation domains and sequences include domains and sequences from the HIV-1-trans-activating protein (Tat), Drosophila Antennapedia homeodomain protein and the herpes simplex-1 virus VP22 protein.

Exogenously added HIV-1-trans-activating protein (Tat) can translocate through the plasma membrane and to reach the nucleus to transactivate the viral genome. Translocational activity has been identified in amino acids 37-72 (Fawell et al., 1994, Proc. Natl. Acad. Sci. U. S. A. 91, 664-668), 37-62 (Anderson et al., 1993, Biochem. Biophys. Res. Commun. 194, 876-884) and 49-58 (having the basic sequence RKKRRQRRR) of HIV-Tat. Vives et al. (1997), J Biol Chem 272, 16010-7 identified a sequence consisting of amino acids 48-60 (CGRKKRRQRRRPPQC), which appears to be important for translocation, nuclear localisation and trans-activation of cellular genes. The third helix of the Drosophila Antennapedia homeodomain protein has also been shown to possess similar properties (reviewed in Prochiantz, A., 1999, Ann N Y Acad Sci, 886, 172-9). The domain responsible for translocation in Antennapedia has been localised to a 16 amino acid long peptide rich in basic amino acids having the sequence RQIKIWFQNRRMKWKK (Derossi, et al., 1994, J Biol Chem, 269, 10444-50). This peptide has been used to direct biologically active substances to the cytoplasm and nucleus of cells in culture (Theodore, et al., 1995, J Neurosci 15, 7158-7167). The VP22 tegument protein of herpes simplex virus is capable of intercellular transport, in which VP22 protein expressed in a subpopulation of cells spreads to other cells in the population (Elliot and O'Hare, 1997, Cell 88, 223-33). Fusion proteins consisting of GFP (Elliott and O'Hare, 1999, Gene Ther 6, 149-51), thymidine kinase protein (Dilber et al., 1999, Gene Ther 6, 12-21) or p53 (Phelan et al., 1998, Nat Biotechnol 16, 440-3) with VP22 have been targeted to cells in this manner. Any of the domains or sequences as set out above may be used to direct RARβ2 or fragment(s) thereof into cell(s). Any of the domains or sequences as set out above, or others identified as having translocational activity, may be used to direct the RARβ2 or fragment(s) thereof into a cell.

Pharmaceutical Compositions

The present invention also provides a pharmaceutical composition comprising administering a therapeutically effective amount of the agent of the present invention (such as RARβ2 and/or an agonist thereof as discussed herein) and a pharmaceutically acceptable carrier, diluent or excipients (including combinations thereof).

1. A viral vector comprising a nucleic acid sequence encoding a receptor.

2. A retroviral vector derived from a lentivirus genome comprising a nucleic acid sequence capable of directing the expression of a receptor.

3. A viral vector comprising a nucleic acid sequence encoding a retinoic acid receptor, preferably retinoic acid receptor β2 (RARβ2).

4. A retroviral vector derived from a lentivirus genome comprising a nucleic acid sequence capable of directing the expression of a retinoic acid receptor, preferably retinoic acid receptor β2 (RARβ2).

5. Use of a vector according to any preceding claim in the preparation of a medicament to cause neurite development.

6. Use of a vector according to any preceding claim in the preparation of a medicament for the treatment of a neurological disorder.

7. A method of treating a neurological disorder comprising administering a vector according to any preceding claim to a subject.

8. A host cell when transduced by a vector according to any preceding claim.

9. A pharmaceutical composition comprising a vector according to any preceding claim in admixture with a pharmaceutically acceptable carrier, diluent or excipient; wherein the pharmaceutical composition is for use to cause neurite development.

10. A retroviral vector derived from a lentivirus genome comprising a nucleic acid sequence capable of directing the expression of at least part of RARβ2 and comprising a deleted gag gene wherein the deletion in gag removes one or more nucleotides downstream of nucleotide 350 of the gag coding sequence.

11. A retroviral vector according to claim 10 wherein the deletion extends from nucleotide 350 to at least the C-terminus of the gag-pol coding region.

12. A retroviral vector according to claim 10 or claim 11 wherein the deletion additionally removes nucleotide 300 of the gag coding region.

13. A retroviral vector according to claim 10 wherein the deletion retains the first 150 nucleotides of the gag coding region.

14. A retroviral vector according to claim 10 wherein the deletion retains the first 109 nucleotides of the gag coding region.

15. A retroviral vector according to claim 10 wherein the deletion retains only the first 2 nucleotides of the gag coding region.

16. A retroviral vector derived from a lentivirus genome wherein one or more accessory genes are absent from the lentivirus genome.

17. A retroviral vector according to claim 16 wherein the accessory genes are selected from dUTPase, S2, rev and tat.

18. A retroviral vector derived from a lentivirus genome wherein the lentivirus genome lacks the tat gene but includes the leader sequences between the end of the 5′ LTR and the ATG of gag.

19. A retroviral vector according to any preceding claim which comprises at least one component from an equine lentivirus.

20. A retroviral vector according to claim 19 wherein the equine lentivirus is EIAV.

21. A retroviral vector according to claim 20 wherein the retroviral vector is substantially derived from EIAV.

22. A method comprising transfecting or transducing a cell with a retroviral vector according to any one of claims 10 to 21.

23. A delivery system in the form of a retroviral vector according to any one of claims 10 to 21.

24. A cell transfected or transduced with a retroviral vector according to any one of claims 10 to 21.

25. Use of a retroviral vector according to any one of claims 10 to 21.

26. Use of a lentiviral gene therapy vector for the delivery of retinoic acid receptor β2 to a cell comprised by the peripheral or central nervous systems.

27. A gene therapy vector comprising a nucleic acid sequence encoding a retinoic acid receptor β2.

28. An EIAV gene therapy vector capable of directing the expression of retinoic acid receptor β2 (RARβ2).

29. A method for producing expression of RARβ2 in an adult mammalian spinal cord cell comprising transducing or transfecting said cell with a vector according to any of claims 10 to 21 or any of claims 27 to 28.

30. A method for stimulation of neurite outgrowth and/or regeneration in a mammalian neuronal cell comprising transducing or transfecting said cell with a vector according to any of claims 10 to 21 or any of claims 27 to 28.

31. Use of a gene therapy vector according to claim 27 or claim 28.

32. A differential expression screening method for identifying genes involved in a cellular process which method comprises comparing gene expression in:
(a) a first cell of interest; and
(b) a second cell of interest which cell comprises altered levels, relative to physiological levels, of a biological molecule due to the introduction into the second cell of a heterologous nucleic acid encoding at least part of RARβ2; and identifying gene products whose expression differs.

33. Use of RARβ2 and/or an agonist thereof in the preparation of a medicament to cause neurite development.

34. Use of RARβ2 and/or an agonist thereof according to claim 33, wherein said agonist is retinoic acid (RA) and/or CD2019.

35. Use of RARβ2 and/or an agonist thereof in the preparation of a medicament for the treatment of a neurological disorder.

36. Use of RARβ2 and/or an agonist thereof according to claim 35, wherein said neurological disorder comprises neurological injury.

37. A method of treating a neurological disorder comprising administering a pharmacologically active amount of an RARβ2 receptor, and/or an agonist thereof.

38. A method according to claim 37, wherein said agonist is RA and/or CD2019.

39. A method according to claim 37 or claim 38, wherein said RARβ2 receptor is administered by an entity comprising a RARβ2 expression system.

40. A method of causing neurite development in a subject, said method comprising providing a nucleic acid construct capable of directing the expression of at least part of a RARβ2 receptor, introducing said construct into one or more cells of said subject, and optionally administering a RARβ2 agonist, such as RA and/or CD2019, to said subject.

41. A pharmaceutical composition comprising RARβ2 and/or an agonist thereof in admixture with a pharmaceutically acceptable carrier, diluent or excipient; wherein the pharmaceutical composition is for use to cause neurite development.

42. Use of a receptor in the production of neurite outgrowth.


SEQUENCE LISTING (73 sequences)

SEQ ID NO: 1 - protein, 9 aa, Human immunodeficiency virus type 1
RKKRRQRRR

SEQ ID NO: 2 - protein, 15 aa, Human immunodeficiency virus type 1
CGRKKRRQRRRPPQC

SEQ ID NO: 3 - protein, 16 aa, Drosophila sp.
RQIKIWFQNRRMKWKK

SEQ ID NO: 4 - DNA, 10998 bp, Artificial Sequence
  Description of Artificial Sequence pONY8.0Z vector genome plasmid
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgacctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 5 - DNA, 12481 bp, Artificial Sequence
  Description of Artificial Sequence pONY3.1, EIAV gag/pol expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgaggtcgacggtatcgcccgaacagggacctgagaggggcgcagaccctacctgttgaacctggctgatcgtaggatccccgggacagcagaggagaacttacagaagtcttctggaggtgttcctggccagaacacaggaggacaggtaagatgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatccaatcatgatagatggggctggaaacagaaattttagacctctaacacctagaggatatactacttgggtgaataccatacagacaaatggtctattaaatgaagctagtcaaaacttatttgggatattatcagtagactgtacttctgaagaaatgaatgcatttttggatgtggtacctggccaggcaggacaaaagcagatattacttgatgcaattgataagatagcagatgattgggataatagacatccattaccgaatgctccactggtggcaccaccacaagggcctattcccatgacagcaaggtttattagaggtttaggagtacctagagaaagacagatggagcctgcttttgatcagtttaggcagacatatagacaatggataatagaagccatgtcagaaggcatcaaagtgatgattggaaaacctaaagctcaaaatattaggcaaggagctaaggaaccttacccagaatttgtagacagactattatcccaaataaaaagtgagggacatccacaagagatttcaaaattcttgactgatacactgactattcagaacgcaaatgaggaatgtagaaatgctatgagacatttaagaccagaggatacattagaagagaaaatgtatgcttgcagagacattggaactacaaaacaaaagatgatgttattggcaaaagcacttcagactggtcttgcgggcccatttaaaggtggagccttgaaaggagggccactaaaggcagcacaaacatgttataactgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatataatctagagaaaaggcctactacaatagtattaattaatgatactcccttaaatgtactgttagacacaggagcagatacttcagtgttgactactgcacattataataggttaaaatatagagggagaaaatatcaagggacgggaataataggagtgggaggaaatgtggaaacattttctacgcctgtgactataaagaaaaagggtagacacattaagacaagaatgctagtggcagatattccagtgactattttgggacgagatattcttcaggacttaggtgcaaaattggttttggcacagctctccaaggaaataaaatttagaaaaatagagttaaaagagggcacaatggggccaaaaattcctcaatggccactcactaaggagaaactagaaggggccaaagagatagtccaaagactattgtcagagggaaaaatatcagaagctagtgacaataatccttataattcacccatatttgtaataaaaaagaggtctggcaaatggaggttattacaagatctgagagaattaaacaaaacagtacaagtaggaacggaaatatccagaggattgcctcacccgggaggattaattaaatgtaaacacatgactgtattagatattggagatgcatatttcactatacccttagatccagagtttagaccatatacagctttcactattccctccattaatcatcaagaaccagataaaagatatgtgtggaaatgtttaccacaaggattcgtgttgagcccatatatatatcagaaaacattacaggaaattttacaaccttttagggaaagatatcctgaagtacaattgtatcaatatatggatgatttgttcatgggaagtaatggttctaaaaaacaacacaaagagttaatcatagaattaagggcgatcttactggaaaagggttttgagacaccagatgataaattacaagaagtgccaccttatagctggctaggttatcaactttgtcctgaaaattggaaagtacaaaaaatgcaattagacatggtaaagaatccaacccttaatgatgtgcaaaaattaatggggaatataacatggatgagctcagggatcccagggttgacagtaaaacacattgcagctactactaagggatgtttagagttgaatcaaaaagtaatttggacggaagaggcacaaaaagagttagaagaaaataatgagaagattaaaaatgctcaagggttacaatattataatccagaagaagaaatgttatgtgaggttgaaattacaaaaaattatgaggcaacttatgttataaaacaatcacaaggaatcctatgggcaggtaaaaagattatgaaggctaataagggatggtcaacagtaaaaaatttaatgttattgttgcaacatgtggcaacagaaagtattactagagtaggaaaatgtccaacgtttaaggtaccatttaccaaagagcaagtaatgtgggaaatgcaaaaaggatggtattattcttggctcccagaaatagtatatacacatcaagtagttcatgatgattggagaatgaaattggtagaagaacctacatcaggaataacaatatacactgatgggggaaaacaaaatggagaaggaatagcagcttatgtgaccagtaatgggagaactaaacagaaaaggttaggacctgtcactcatcaagttgctgaaagaatggcaatacaaatggcattagaggataccagagataaacaagtaaatatagtaactgatagttattattgttggaaaaatattacagaaggattaggtttagaaggaccacaaagtccttggtggcctataatacaaaatatacgagaaaaagagatagtttattttgcttgggtacctggtcacaaagggatatatggtaatcaattggcagatgaagccgcaaaaataaaagaagaaatcatgctagcataccaaggcacacaaattaaagagaaaagagatgaagatgcagggtttgacttatgtgttccttatgacatcatgatacctgtatctgacacaaaaatcatacccacagatgtaaaaattcaagttcctcctaatagctttggatgggtcactgggaaatcatcaatggcaaaacaggggttattaattaatggaggaataattgatgaaggatatacaggagaaatacaagtgatatgtactaatattggaaaaagtaatattaaattaatagagggacaaaaatttgcacaattaattatactacagcatcactcaaattccagacagccttgggatgaaaataaaatatctcagagaggggataaaggatttggaagtacaggagtattctgggtagaaaatattcaggaagcacaagatgaacatgagaattggcatacatcaccaaagatattggcaagaaattataagataccattgactgtagcaaaacagataactcaagaatgtcctcattgcactaagcaaggatcaggacctgcaggttgtgtcatgagatctcctaatcattggcaggcagattgcacacatttggacaataagataatattgacttttgtagagtcaaattcaggatacatacatgctacattattgtcaaaagaaaatgcattatgtacttcattggctattttagaatgggcaagattgttttcaccaaagtccttacacacagataacggcactaattttgtggcagaaccagttgtaaatttgttgaagttcctaaagatagcacataccacaggaataccatatcatccagaaagtcagggtattgtagaaagggcaaataggaccttgaaagagaagattcaaagtcatagagacaacactcaaacactggaggcagctttacaacttgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgaggtctctaggaattgattacctcgatgcttcattaaggaagaagaataaacaaagactgaaggcaatccaacaaggaagacaacctcaatatttgttataaggtttgatatatgggagtatttggtaaaggggtaacatggtcagcatcgcattctatgggggaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 6 - DNA, 6845 bp, Artificial Sequence
  Description of Artificial Sequence pRV67, VSV-G expression plasmid
tcgacctgcaggatatcgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgtaccgagctcgcgtaatcatgtcatagctgtttcctgtgtgaaattgttatccgctcacaattccacacaacatacgagccggaagcataaagtgtaaagcctggggtgcctaatgagtgagctaactacattaattgcgttgcgctcackgcccgctttccartcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacmctagaagracagtatttggkatctgsgcttctgytgaagmcagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaataagcggccgcggccatgccggccactagtctcgagttattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgaagacgtcgcctcctcactacttctggaatagctcagaggccgaggcggcctcggcctctgcataaataaaaaaaatwaktcasggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccatcgtgtcaaaggacagtgactgcagtgaataataaaatgtgtgtttgtccgaaatacgcgttttgagawttctgtcgccgactaaattcatgtcgcgcgatartggtgtttatcgccgatagagatggcgatattggaaaaatcgatatttgaaaatatggcatattgaaaatgtcgccgatgtgagtttctgtgtaactgatatcgccatttttccaaaagttgatttttgggcatacgcgatatctggcgatacgcttatatcgtttacgggggatggcgatagacgcctttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgcacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccagcgaatctcgggtacgtgttccggacatggggctcttctccggtagcggsggagyttctacatccgrrsccttgytcccatgcctccasgrmttcatgktcgytcggcagctccttgctcctaamcagtggaggccagacttaggcacagcacgatgcccaccaccaccagtgtgccgcacaaggccgtggcggtagggtatgtgtctgaaaatgagctcggggagcgggcttgcaccgctggacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttcccgggataggtacctcgcgagatccctcgaggaggaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatgggggcctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcctcgagggatctagag

SEQ ID NO: 7 - DNA, 1375 bp, Artificial Sequence
  Description of Artificial Sequence RARbeta2 PCR product
actgccgcgggccaccatgtttgactgtatggatgttctgtcagtgagtcccgggcagatcctggatttctacaccgcgagcccttcctcctgcatgctgcaggaaaaggctctcaaagcctgcctcagtggattcacccaggccgaatggcagcaccggcatactgctcaatccatcgagacacagagtaccagctctgaggagctcgtcccgagcccaccatctccacttcctcctcctcgggtgtacaagccctgcttcgtttgccaggacaagtcatcgggctaccactatggcgtcagtgcctgcgaggggtgcaagggctttttccgcagaagtattcagaagaacatgatctacacttgccatcgagataagaactgcgtcattaacaaggtcactaggaaccgatgccagtactgccgcctgcagaagtgctttgaagtgggcatgtccaaagagtctgttaggaatgacaggaacaagaaaaagaaggagccttcaaagcaggaatgcacagagagctatgagatgacagcggagctagacgacctcactgagaagatccggaaagcccaccaggaaacctttccctcactctgccagctgggtaaatacaccacgaattccagcgctgaccaccgggtccgattggacttgggcctctgggacaaattcagtgagctggccaccaagtgcattattaagatcgtggagttcgccaagcgtctgccgggcttcacaggtctgaccatcgcagaccagatcaccctgctcaaagccgcctgcttggatatcttgattctcagaatttgtaccaggtataccccagagcaagacaccatgactttctctgatggccttacactaaatcgaactcagatgcacaatgctggcttcggtcctctgactgaccttgtgttcacctttgccaaccagctcctgcctttggaaatggatgacacagaaacaggccttctcagtgccatctgtttaatctgtggagaccgccaggaccttgaggaaccaacaaaagtagacaagctccaagaaccactgctggaagcactaaagatttacattagaaaacgacgacccagcaagcctcacatgtttccaaagatcttaatgaaaatcacagatctccgcagcatcagcgcgaaaggtgccgaacgtgtaattaccttgaaaatggaaattcctggatcaatgccacctctcattcaggaaatgctggagaattctgaaggacatgaacccttgaccccaagttcaagtgggaatatagcagagcacagtcccagcgtgtcccccagctcagtggagaacagtggagtcagtcagtcaccactgctgcagtgagcggccgccagt

SEQ ID NO: 8 - DNA, 1399 bp, Artificial Sequence
  Description of Artificial Sequence FLAG RARbeta2 PCR product
actgccgcgggccaccatggactacaaggacgacgatgacaagtttgactgtatggatgttctgtcagtgagtcccgggcagatcctggatttctacaccgcgagcccttcctcctgcatgctgcaggaaaaggctctcaaagcctgcctcagtggattcacccaggccgaatggcagcaccggcatactgctcaatccatcgagacacagagtaccagctctgaggagctcgtcccgagcccaccatctccacttcctcctcctcgggtgtacaagccctgcttcgtttgccaggacaagtcatcgggctaccactatggcgtcagtgcctgcgaggggtgcaagggctttttccgcagaagtattcagaagaacatgatctacacttgccatcgagataagaactgcgtcattaacaaggtcactaggaaccgatgccagtactgccgcctgcagaagtgctttgaagtgggcatgtccaaagagtctgttaggaatgacaggaacaagaaaaagaaggagccttcaaagcaggaatgcacagagagctatgagatgacagcggagctagacgacctcactgagaagatccggaaagcccaccaggaaacctttccctcactctgccagctgggtaaatacaccacgaattccagcgctgaccaccgggtccgattggacttgggcctctgggacaaattcagtgagctggccaccaagtgcattattaagatcgtggagttcgccaagcgtctgccgggcttcacaggtctgaccatcgcagaccagatcaccctgctcaaagccgcctgcttggatatcttgattctcagaatttgtaccaggtataccccagagcaagacaccatgactttctctgatggccttacactaaatcgaactcagatgcacaatgctggcttcggtcctctgactgaccttgtgttcacctttgccaaccagctcctgcctttggaaatggatgacacagaaacaggccttctcagtgccatctgtttaatctgtggagaccgccaggaccttgaggaaccaacaaaagtagacaagctccaagaaccactgctggaagcactaaagatttacattagaaaacgacgacccagcaagcctcacatgtttccaaagatcttaatgaaaatcacagatctccgcagcatcagcgcgaaaggtgccgaacgtgtaattaccttgaaaatggaaattcctggatcaatgccacctctcattcaggaaatgctggagaattctgaaggacatgaacccttgaccccaagttcaagtgggaatatagcagagcacagtcccagcgtgtcccccagctcagtggagaacagtggagtcagtcagtcaccactgctgcagtgagcggccgccagt

SEQ ID NO: 9 - DNA, 9127 bp, Artificial Sequence
  Description of Artificial Sequence pONY- RARbeta2 vector genome plasmid
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagagtcgacgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatggtcgactctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcgggccaccatgtttgactgtatggatgttctgtcagtgagtcccgggcagatcctggatttctacaccgcgagcccttcctcctgcatgctgcaggaaaaggctctcaaagcctgcctcagtggattcacccaggccgaatggcagcaccggcatactgctcaatccatcgagacacagagtaccagctctgaggagctcgtcccgagcccaccatctccacttcctcctcctcgggtgtacaagccctgcttcgtttgccaggacaagtcatcgggctaccactatggcgtcagtgcctgcgaggggtgcaagggctttttccgcagaagtattcagaagaacatgatctacacttgccatcgagataagaactgcgtcattaacaaggtcactaggaaccgatgccagtactgccgcctgcagaagtgctttgaagtgggcatgtccaaagagtctgttaggaatgacaggaacaagaaaaagaaggagccttcaaagcaggaatgcacagagagctatgagatgacagcggagctagacgacctcactgagaagatccggaaagcccaccaggaaacctttccctcactctgccagctgggtaaatacaccacgaattccagcgctgaccaccgggtccgattggacttgggcctctgggacaaattcagtgagctggccaccaagtgcattattaagatcgtggagttcgccaagcgtctgccgggcttcacaggtctgaccatcgcagaccagatcaccctgctcaaagccgcctgcttggatatcttgattctcagaatttgtaccaggtataccccagagcaagacaccatgactttctctgatggccttacactaaatcgaactcagatgcacaatgctggcttcggtcctctgactgaccttgtgttcacctttgccaaccagctcctgcctttggaaatggatgacacagaaacaggccttctcagtgccatctgtttaatctgtggagaccgccaggaccttgaggaaccaacaaaagtagacaagctccaagaaccactgctggaagcactaaagatttacattagaaaacgacgacccagcaagcctcacatgtttccaaagatcttaatgaaaatcacagatctccgcagcatcagcgcgaaaggtgccgaacgtgtaattaccttgaaaatggaaattcctggatcaatgccacctctcattcaggaaatgctggagaattctgaaggacatgaacccttgaccccaagttcaagtgggaatatagcagagcacagtcccagcgtgtcccccagctcagtggagaacagtggagtcagtcagtcaccactgctgcagtgagcggccgcgactctagagtcgacctcgagggggggcccggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 10 - DNA, 9151 bp, Artificial Sequence
  Description of Artificial Sequence pONY-FLAG-RARbeta2 vector genome plasmid
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagagtcgacgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatggtcgactctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcgggccaccatggactacaaggacgacgatgacaagtttgactgtatggatgttctgtcagtgagtcccgggcagatcctggatttctacaccgcgagcccttcctcctgcatgctgcaggaaaaggctctcaaagcctgcctcagtggattcacccaggccgaatggcagcaccggcatactgctcaatccatcgagacacagagtaccagctctgaggagctcgtcccgagcccaccatctccacttcctcctcctcgggtgtacaagccctgcttcgtttgccaggacaagtcatcgggctaccactatggcgtcagtgcctgcgaggggtgcaagggctttttccgcagaagtattcagaagaacatgatctacacttgccatcgagataagaactgcgtcattaacaaggtcactaggaaccgatgccagtactgccgcctgcagaagtgctttgaagtgggcatgtccaaagagtctgttaggaatgacaggaacaagaaaaagaaggagccttcaaagcaggaatgcacagagagctatgagatgacagcggagctagacgacctcactgagaagatccggaaagcccaccaggaaacctttccctcactctgccagctgggtaaatacaccacgaattccagcgctgaccaccgggtccgattggacttgggcctctgggacaaattcagtgagctggccaccaagtgcattattaagatcgtggagttcgccaagcgtctgccgggcttcacaggtctgaccatcgcagaccagatcaccctgctcaaagccgcctgcttggatatcttgattctcagaatttgtaccaggtataccccagagcaagacaccatgactttctctgatggccttacactaaatcgaactcagatgcacaatgctggcttcggtcctctgactgaccttgtgttcacctttgccaaccagctcctgcctttggaaatggatgacacagaaacaggccttctcagtgccatctgtttaatctgtggagaccgccaggaccttgaggaaccaacaaaagtagacaagctccaagaaccactgctggaagcactaaagatttacattagaaaacgacgacccagcaagcctcacatgtttccaaagatcttaatgaaaatcacagatctccgcagcatcagcgcgaaaggtgccgaacgtgtaattaccttgaaaatggaaattcctggatcaatgccacctctcattcaggaaatgctggagaattctgaaggacatgaacccttgaccccaagttcaagtgggaatatagcagagcacagtcccagcgtgtcccccagctcagtggagaacagtggagtcagtcagtcaccactgctgcagtgagcggccgcgactctagagtcgacctcgagggggggcccggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 11 - DNA, 8528 bp, Artificial Sequence
  Description of Artificial Sequence pONY8G 5′cPPT POS delCTS EIAV vector genome plasmid
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagagtcgacgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatggtcgactctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttgttgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctcgagggggggcccggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgacctcaatatttgttataaggtttgatatgaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 12 - DNA, 10112 bp, Artificial Sequence
  Description of Artificial Sequence pESYNGP, codon-optimised EIAV gag/pol expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagaattcgccaccatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccggggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 13 - DNA, 10114 bp, Artificial Sequence
  Description of Artificial Sequence pESDSYNGP, codon-optimised EIAV gag/pol expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagaattccaggtaagatgggcgatcccctcacctggtccaaagccctgaagaaactggaaaaagtcaccgttcagggtagccaaaagcttaccacaggcaattgcaactgggcattgtccctggtggatcttttccacgacactaatttcgttaaggagaaagattggcaactcagagacgtgatccccctcttggaggacgtgacccaaacattgtctgggcaggagcgcgaagctttcgagcgcacctggtgggccatcagcgcagtcaaaatggggctgcaaatcaacaacgtggttgacggtaaagctagctttcaactgctccgcgctaagtacgagaagaaaaccgccaacaagaaacaatccgaacctagcgaggagtacccaattatgatcgacggcgccggcaataggaacttccgcccactgactcccaggggctataccacctgggtcaacaccatccagacaaacggacttttgaacgaagcctcccagaacctgttcggcatcctgtctgtggactgcacctccgaagaaatgaatgcttttctcgacgtggtgccaggacaggctggacagaaacagatcctgctcgatgccattgacaagatcgccgacgactgggataatcgccaccccctgccaaacgcccctctggtggctcccccacaggggcctatccctatgaccgctaggttcattaggggactgggggtgccccgcgaacgccagatggagccagcatttgaccaatttaggcagacctacagacagtggatcatcgaagccatgagcgaggggattaaagtcatgatcggaaagcccaaggcacagaacatcaggcagggggccaaggaaccataccctgagtttgtcgacaggcttctgtcccagattaaatccgaaggccaccctcaggagatctccaagttcttgacagacacactgactatccaaaatgcaaatgaagagtgcagaaacgccatgaggcacctcagacctgaagataccctggaggagaaaatgtacgcatgtcgcgacattggcactaccaagcaaaagatgatgctgctcgccaaggctctgcaaaccggcctggctggtccattcaaaggaggagcactgaagggaggtccattgaaagctgcacaaacatgttataattgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatacaatctcgagaagaggcccactaccatcgtcctgatcaatgacacccctcttaatgtgctgctggacaccggagccgacaccagcgttctcactactgctcactataacagactgaaatacagaggaaggaaataccagggcacaggcatcatcggcgttggaggcaacgtcgaaaccttttccactcctgtcaccatcaaaaagaaggggagacacattaaaaccagaatgctggtcgccgacatccccgtcaccatccttggcagagacattctccaggacctgggcgctaaactcgtgctggcacaactgtctaaggaaatcaagttccgcaagatcgagctgaaagagggcacaatgggtccaaaaatcccccagtggcccctgaccaaagagaagcttgagggcgctaaggaaatcgtgcagcgcctgctttctgagggcaagattagcgaggccagcgacaataacccttacaacagccccatctttgtgattaagaaaaggagcggcaaatggagactcctgcaggacctgagggaactcaacaagaccgtccaggtcggaactgagatctctcgcggactgcctcaccccggcggcctgattaaatgcaagcacatgacagtccttgacattggagacgcttattttaccatccccctcgatcctgaatttcgcccctatactgcttttaccatccccagcatcaatcaccaggagcccgataaacgctatgtgtggaagtgcctcccccagggatttgtgcttagcccctacatttaccagaagacacttcaagagatcctccaacctttccgcgaaagatacccagaggttcaactctaccaatatatggacgacctgttcatggggtccaacgggtctaagaagcagcacaaggaactcatcatcgaactgagggcaatcctcctggagaaaggcttcgagacacccgacgacaagctgcaagaagttcctccatatagctggctgggctaccagctttgccctgaaaactggaaagtccagaagatgcagttggatatggtcaagaacccaacactgaacgacgtccagaagctcatgggcaatattacctggatgagctccggaatccctgggcttaccgttaagcacattgccgcaactacaaaaggatgcctggagttgaaccagaaggtcatttggacagaggaagctcagaaggaactggaggagaataatgaaaagattaagaatgctcaagggctccaatactacaatcccgaagaagaaatgttgtgcgaggtcgaaatcactaagaactacgaagccacctatgtcatcaaacagtcccaaggcatcttgtgggccggaaagaaaatcatgaaggccaacaaaggctggtccaccgttaaaaatctgatgctcctgctccagcacgtcgccaccgagtctatcacccgcgtcggcaagtgccccaccttcaaagttcccttcactaaggagcaggtgatgtgggagatgcaaaaaggctggtactactcttggcttcccgagatcgtctacacccaccaagtggtgcacgacgactggagaatgaagcttgtcgaggagcccactagcggaattacaatctataccgacggcggaaagcaaaacggagagggaatcgctgcatacgtcacatctaacggccgcaccaagcaaaagaggctcggccctgtcactcaccaggtggctgagaggatggctatccagatggcccttgaggacactagagacaagcaggtgaacattgtgactgacagctactactgctggaaaaacatcacagagggccttggcctggagggaccccagtctccctggtggcctatcatccagaatatccgcgaaaaggaaattgtctatttcgcctgggtgcctggacacaaaggaatttacggcaaccaactcgccgatgaagccgccaaaattaaagaggaaatcatgcttgcctaccagggcacacagattaaggagaagagagacgaggacgctggctttgacctgtgtgtgccatacgacatcatgattcccgttagcgacacaaagatcattccaaccgatgtcaagatccaggtgccacccaattcatttggttgggtgaccggaaagtccagcatggctaagcagggtcttctgattaacgggggaatcattgatgaaggatacaccggcgaaatccaggtgatctgcacaaatatcggcaaaagcaatattaagcttatcgaagggcagaagttcgctcaactcatcatcctccagcaccacagcaattcaagacaaccttgggacgaaaacaagattagccagagaggtgacaagggcttcggcagcacaggtgtgttctgggtggagaacatccaggaagcacaggacgagcacgagaattggcacacctcccctaagattttggcccgcaattacaagatcccactgactgtggctaagcagatcacacaggaatgcccccactgcaccaaacaaggttctggccccgccggctgcgtgatgaggtcccccaatcactggcaggcagattgcacccacctcgacaacaaaattatcctgaccttcgtggagagcaattccggctacatccacgcaacactcctctccaaggaaaatgcattgtgcacctccctcgcaattctggaatgggccaggctgttctctccaaaatccctgcacaccgacaacggcaccaactttgtggctgaacctgtggtgaatctgctgaagttcctgaaaatcgcccacaccactggcattccctatcaccctgaaagccagggcattgtcgagagggccaacagaactctgaaagaaaagatccaatctcacagagacaatacacagacattggaggccgcacttcagctcgcccttatcacctgcaacaaaggaagagaaagcatgggcggccagaccccctgggaggtcttcatcactaaccaggcccaggtcatccatgaaaagctgctcttgcagcaggcccagtcctccaaaaagttctgcttttataagatccccggtgagcacgactggaaaggtcctacaagagttttgtggaaaggagacggcgcagttgtggtgaacgatgagggcaaggggatcatcgctgtgcccctgacacgcaccaagcttctcatcaagccaaactgaacccggggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 14 - DNA, 5993 bp, Artificial Sequence
  Description of Artificial Sequence pClneoERev, EIAV Rev expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagtaacggccgccagtgtgctggaattcggcttatggcagaatcgaaggaagcaagagaccaagaaatgaacctgaaagaagaatctaaagaagaaaaaagaagaaatgactggtggaaaatagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgaagccgaattctgcagatatccatcacactggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 15 - DNA, 5961 bp, Artificial Sequence
  Description of Artificial Sequence pESYNREV, codon-optimised EIAV Rev expression plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgccaccatggctgagagcaaggaggccagggatcaagagatgaacctcaaggaagagagcaaagaggagaagcgccgcaacgactggtggaagatcgacccacaaggccccctggagggggaccagtggtgccgcgtgctgagacagtccctgcccgaggagaagattcctagccagacctgcatcgccagaagacacctcggccccggtcccacccagcacacaccctccagaagggataggtggattaggggccagattttgcaagccgaggtcctccaagaaaggctggaatggagaattaggggcgtgcaacaagccgctaaagagctgggagaggtgaatcgcggcatctggagggagctctacttccgcgaggaccagaggggcgatttctccgcatggggaggctaccagagggcacaagaaaggctgtggggcgagcagagcagcccccgcgtcttgaggcccggagactccaaaagacgccgcaaacacctgtgaagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 16 - DNA, 42 bp, Artificial Sequence
  Description of Artificial Sequence Primer
ggctagagaattccaggtaagatgggcgatcccctcacctgg

SEQ ID NO: 17 - DNA, 22 bp, Artificial Sequence
  Description of Artificial Sequence Primer
ttgggtactcctcgctaggttc

SEQ ID NO: 18 - DNA, 21 bp, Artificial Sequence
  Description of Artificial Sequence Sequence flanking the EIAV gag/pol ORF
tctagagaattcgccaccatg

SEQ ID NO: 19 - RNA, 18 nt, Artificial Sequence
  Description of Artificial Sequence Sequence flanking the EIAV gag/pol ORF
ugaacccggggcggccgc

SEQ ID NO: 20 - DNA, 42 bp, Artificial Sequence
  Description of Artificial Sequence Primer
cagtacccgcgggccaccatgtttgactgtatggatgttctg

SEQ ID NO: 21 - DNA, 37 bp, Artificial Sequence
  Description of Artificial Sequence Primer
cagtacctgcagatcattgcacgagtggtgactgact

SEQ ID NO: 22 - DNA, 38 bp, Artificial Sequence
  Description of Artificial Sequence Primer
caggttattctagagtcgacgctctcattacttgtaac

SEQ ID NO: 23 - DNA, 41 bp, Artificial Sequence
  Description of Artificial Sequence Primer
cgaatgcgttctagagtcgaccatgttcaccagggattttg

SEQ ID NO: 24 - DNA, 23 bp, Artificial Sequence
  Description of Artificial Sequence Primer
cacctagcaggcgtgaccggtgg

SEQ ID NO: 25 - DNA, 37 bp, Artificial Sequence
  Description of Artificial Sequence Primer
cctaccaattgtataaaacccctcataaaaaccccac

SEQ ID NO: 26 - DNA, 24 bp, Artificial Sequence
  Description of Artificial Sequence Primer
cacaggtcaaacctcctaggaatg

SEQ ID NO: 27 - DNA, 21 bp, Artificial Sequence
  Description of Artificial Sequence Primer
tcctgctcaacttcctgtcga

SEQ ID NO: 28 - DNA, 19 bp, Artificial Sequence
  Description of Artificial Sequence Probe
cgagacgctaccatggcta

SEQ ID NO: 29 - DNA, 29 bp, Artificial Sequence
  Description of Artificial Sequence Primer
accagtagttaatttctgagacccttgta

SEQ ID NO: 30 - DNA, 21 bp, Artificial Sequence
  Description of Artificial Sequence Primer
attgggagaccctttgacatt

SEQ ID NO: 31 - DNA, 30 bp, Artificial Sequence
  Description of Artificial Sequence Probe
caccttctctaacttcttgagcgccttgct

SEQ ID NO: 32 - DNA, 42 bp, Artificial Sequence
  Description of Artificial Sequence Primer
actgccgcgggccaccatgtttgactgtatggatgttctgtc

SEQ ID NO: 33 - DNA, 66 bp, Artificial Sequence
  Description of Artificial Sequence Primer
actgccgcgggccaccatggactacaaggacgacgatgacaagtttgactgtatggatgttctgtc

SEQ ID NO: 34 - DNA, 29 bp, Artificial Sequence
  Description of Artificial Sequence Primer
actggcggccgctcactgcagcagtggtg

SEQ ID NO: 35 - DNA, 38 bp, Artificial Sequence; identical to SEQ ID NO: 22
  Description of Artificial Sequence Primer

SEQ ID NO: 36 - DNA, 41 bp, Artificial Sequence; identical to SEQ ID NO: 23
  Description of Artificial Sequence Primer

SEQ ID NO: 37 - DNA, 19 bp, Artificial Sequence
  Description of Artificial Sequence Primer
tgtagctctctgagcactc

SEQ ID NO: 38 - DNA, 19 bp, Artificial Sequence
  Description of Artificial Sequence Primer
tacgccttcttctttcccc

SEQ ID NO: 39 - DNA, 21 bp, Artificial Sequence
  Description of Artificial Sequence Primer
cttttataaccagaaccgggc

SEQ ID NO: 40 - DNA, 21 bp, Artificial Sequence
  Description of Artificial Sequence Primer
caagtagaagccaggaaagtc

SEQ ID NO: 41 - DNA, 21 bp, Artificial Sequence
  Description of Artificial Sequence Primer
ctaagaagacccacacttctg

SEQ ID NO: 42 - DNA, 19 bp, Artificial Sequence
  Description of Artificial Sequence Primer
aagtgaggtgaaaactggg

SEQ ID NO: 43 - DNA, 18 bp, Artificial Sequence
  Description of Artificial Sequence Primer
ttcacagcctggcataac

SEQ ID NO: 44 - DNA, 19 bp, Artificial Sequence
  Description of Artificial Sequence Primer
gagaaggaagtgagccatc

SEQ ID NO: 45 - DNA, 21 bp, Artificial Sequence
  Description of Artificial Sequence Primer
tctctgtgcattcctgctttg

SEQ ID NO: 46 - DNA, 20 bp, Artificial Sequence
  Description of Artificial Sequence Primer
tggacacatgactcactacc

SEQ ID NO: 47 - DNA, 20 bp, Artificial Sequence
  Description of Artificial Sequence Primer
atgttctgtcagtgagtccc

SEQ ID NO: 48 - DNA, 19 bp, Artificial Sequence
  Description of Artificial Sequence Primer
gcatgtcagaggacaactg

SEQ ID NO: 49 - DNA, 18 bp, Artificial Sequence
  Description of Artificial Sequence Primer
agcctggaaaatgccatc

SEQ ID NO: 50 - DNA, 22 bp, Artificial Sequence
  Description of Artificial Sequence Primer
ttacagcttccttggacatgcc

SEQ ID NO: 51 - DNA, 20 bp, Artificial Sequence
  Description of Artificial Sequence Primer
agatgctgagccctagcttc

SEQ ID NO: 52 - DNA, 20 bp, Artificial Sequence
  Description of Artificial Sequence Primer
ttactacgcagagccactgg

SEQ ID NO: 53 - DNA, 19 bp, Artificial Sequence
  Description of Artificial Sequence Primer
ggaagatggaagagggaac

SEQ ID NO: 54 - DNA, 19 bp, Artificial Sequence
  Description of Artificial Sequence Primer
caaatttactgggggttgg

SEQ ID NO: 55 - DNA, 20 bp, Artificial Sequence
  Description of Artificial Sequence Primer
ggctggattttggattgaag

SEQ ID NO: 56 - DNA, 22 bp, Artificial Sequence
  Description of Artificial Sequence Primer
ttctgtcctctcactaccttgg

SEQ ID NO: 57 - DNA, 20 bp, Artificial Sequence
  Description of Artificial Sequence Primer
cattaccgcgagtcactaac

SEQ ID NO: 58 - DNA, 20 bp, Artificial Sequence
  Description of Artificial Sequence Primer
cgtagacaaaatggtgaagg

SEQ ID NO: 59 - DNA, 20 bp, Artificial Sequence
  Description of Artificial Sequence Primer
gactccacgacatactcagc

SEQ ID NO: 60 - DNA, 18 bp, Artificial Sequence
  Description of Artificial Sequence Primer
gcttcttcattgacccac

SEQ ID NO: 61 - DNA, 20 bp, Artificial Sequence
  Description of Artificial Sequence Primer
cttcaccgtcaggtctttac

SEQ ID NO: 62 - DNA, 20 bp, Artificial Sequence
  Description of Artificial Sequence Primer
gcaaggaccggaatgagaac

SEQ ID NO: 63 - DNA, 21 bp, Artificial Sequence
  Description of Artificial Sequence Primer
tctaggggcagctcagaaaag

SEQ ID NO: 64 - DNA, 21 bp, Artificial Sequence
  Description of Artificial Sequence Primer
agaataaaggggtagtgaagg

SEQ ID NO: 65 - DNA, 18 bp, Artificial Sequence
  Description of Artificial Sequence Primer
catcaatgtccccacttg

SEQ ID NO: 66 - DNA, 20 bp, Artificial Sequence
  Description of Artificial Sequence Primer
tgccagtagtagccacgaag

SEQ ID NO: 67 - DNA, 19 bp, Artificial Sequence
  Description of Artificial Sequence Primer
tgagcagttcattccaccc

SEQ ID NO: 68 - DNA, 10998 bp, Artificial Sequence; identical to SEQ ID NO: 4
  Description of Artificial Sequence pONY8.0Z

SEQ ID NO: 69 - DNA, 12481 bp, Artificial Sequence
  Description of Artificial Sequence pONY3.1
agatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgaggtcgacggtatcgcccgaacagggacctgagaggggcgcagaccctacctgttgaacctggctgatcgtaggatccccgggacagcagaggagaacttacagaagtcttctggaggtgttcctggccagaacacaggaggacaggtaagatgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatccaatcatgatagatggggctggaaacagaaattttagacctctaacacctagaggatatactacttgggtgaataccatacagacaaatggtctattaaatgaagctagtcaaaacttatttgggatattatcagtagactgtacttctgaagaaatgaatgcatttttggatgtggtacctggccaggcaggacaaaagcagatattacttgatgcaattgataagatagcagatgattgggataatagacatccattaccgaatgctccactggtggcaccaccacaagggcctattcccatgacagcaaggtttattagaggtttaggagtacctagagaaagacagatggagcctgcttttgatcagtttaggcagacatatagacaatggataatagaagccatgtcagaaggcatcaaagtgatgattggaaaacctaaagctcaaaatattaggcaaggagctaaggaaccttacccagaatttgtagacagactattatcccaaataaaaagtgagggacatccacaagagatttcaaaattcttgactgatacactgactattcagaacgcaaatgaggaatgtagaaatgctatgagacatttaagaccagaggatacattagaagagaaaatgtatgcttgcagagacattggaactacaaaacaaaagatgatgttattggcaaaagcacttcagactggtcttgcgggcccatttaaaggtggagccttgaaaggagggccactaaaggcagcacaaacatgttataactgtgggaagccaggacatttatctagtcaatgtagagcacctaaagtctgttttaaatgtaaacagcctggacatttctcaaagcaatgcagaagtgttccaaaaaacgggaagcaaggggctcaagggaggccccagaaacaaactttcccgatacaacagaagagtcagcacaacaaatctgttgtacaagagactcctcagactcaaaatctgtacccagatctgagcgaaataaaaaaggaatacaatgtcaaggagaaggatcaagtagaggatctcaacctggacagtttgtgggagtaacatataatctagagaaaaggcctactacaatagtattaattaatgatactcccttaaatgtactgttagacacaggagcagatacttcagtgttgactactgcacattataataggttaaaatatagagggagaaaatatcaagggacgggaataataggagtgggaggaaatgtggaaacattttctacgcctgtgactataaagaaaaagggtagacacattaagacaagaatgctagtggcagatattccagtgactattttgggacgagatattcttcaggacttaggtgcaaaattggttttggcacagctctccaaggaaataaaatttagaaaaatagagttaaaagagggcacaatggggccaaaaattcctcaatggccactcactaaggagaaactagaaggggccaaagagatagtccaaagactattgtcagagggaaaaatatcagaagctagtgacaataatccttataattcacccatatttgtaataaaaaagaggtctggcaaatggaggttattacaagatctgagagaattaaacaaaacagtacaagtaggaacggaaatatccagaggattgcctcacccgggaggattaattaaatgtaaacacatgactgtattagatattggagatgcatatttcactatacccttagatccagagtttagaccatatacagctttcactattccctccattaatcatcaagaaccagataaaagatatgtgtggaaatgtttaccacaaggattcgtgttgagcccatatatatatcagaaaacattacaggaaattttacaaccttttagggaaagatatcctgaagtacaattgtatcaatatatggatgatttgttcatgggaagtaatggttctaaaaaacaacacaaagagttaatcatagaattaagggcgatcttactggaaaagggttttgagacaccagatgataaattacaagaagtgccaccttatagctggctaggttatcaactttgtcctgaaaattggaaagtacaaaaaatgcaattagacatggtaaagaatccaacccttaatgatgtgcaaaaattaatggggaatataacatggatgagctcagggatcccagggttgacagtaaaacacattgcagctactactaagggatgtttagagttgaatcaaaaagtaatttggacggaagaggcacaaaaagagttagaagaaaataatgagaagattaaaaatgctcaagggttacaatattataatccagaagaagaaatgttatgtgaggttgaaattacaaaaaattatgaggcaacttatgttataaaacaatcacaaggaatcctatgggcaggtaaaaagattatgaaggctaataagggatggtcaacagtaaaaaatttaatgttattgttgcaacatgtggcaacagaaagtattactagagtaggaaaatgtccaacgtttaaggtaccatttaccaaagagcaagtaatgtgggaaatgcaaaaaggatggtattattcttggctcccagaaatagtatatacacatcaagtagttcatgatgattggagaatgaaattggtagaagaacctacatcaggaataacaatatacactgatgggggaaaacaaaatggagaaggaatagcagcttatgtgaccagtaatgggagaactaaacagaaaaggttaggacctgtcactcatcaagttgctgaaagaatggcaatacaaatggcattagaggataccagagataaacaagtaaatatagtaactgatagttattattgttggaaaaatattacagaaggattaggtttagaaggaccacaaagtccttggtggcctataatacaaaatatacgagaaaaagagatagtttattttgcttgggtacctggtcacaaagggatatatggtaatcaattggcagatgaagccgcaaaaataaaagaagaaatcatgctagcataccaaggcacacaaattaaagagaaaagagatgaagatgcagggtttgacttatgtgttccttatgacatcatgatacctgtatctgacacaaaaatcatacccacagatgtaaaaattcaagttcctcctaatagctttggatgggtcactgggaaatcatcaatggcaaaacaggggttattaattaatggaggaataattgatgaaggatatacaggagaaatacaagtgatatgtactaatattggaaaaagtaatattaaattaatagagggacaaaaatttgcacaattaattatactacagcatcactcaaattccagacagccttgggatgaaaataaaatatctcagagaggggataaaggatttggaagtacaggagtattctgggtagaaaatattcaggaagcacaagatgaacatgagaattggcatacatcaccaaagatattggcaagaaattataagataccattgactgtagcaaaacagataactcaagaatgtcctcattgcactaagcaaggatcaggacctgcaggttgtgtcatgagatctcctaatcattggcaggcagattgcacacatttggacaataagataatattgacttttgtagagtcaaattcaggatacatacatgctacattattgtcaaaagaaaatgcattatgtacttcattggctattttagaatgggcaagattgttttcaccaaagtccttacacacagataacggcactaattttgtggcagaaccagttgtaaatttgttgaagttcctaaagatagcacataccacaggaataccatatcatccagaaagtcagggtattgtagaaagggcaaataggaccttgaaagagaagattcaaagtcatagagacaacactcaaacactggaggcagctttacaacttgctctcattacttgtaacaaagggagggaaagtatgggaggacagacaccatgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgaggtctctaggaattgattacctcgatgcttcattaaggaagaagaataaacaaagactgaaggcaatccaacaaggaagacaacctcaatatttgttataaggtttgatatatgggagtatttggtaaaggggtaacatggtcagcatcgcattctatgggggaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 70 - DNA, 10112 bp, Artificial Sequence; identical to SEQ ID NO: 12
  Description of Artificial Sequence pEsynGP

SEQ ID NO: 71 - DNA, 10114 bp, Artificial Sequence; identical to SEQ ID NO: 13
  Description of Artificial Sequence pESDSYNGP

SEQ ID NO: 72 - DNA, 11131 bp, Artificial Sequence
  Description of Artificial Sequence pONY4.0Z
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggcagatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagatgggagaccctttgacatggagcaaggcgctcaagaagttagagaaggtgacggtacaagggtctcagaaattaactactggtaactgtaattgggcgctaagtctagtagacttatttcatgataccaactttgtaaaagaaaaggactggcagctgagggatgtcattccattgctggaagatgtaactcagacgctgtcaggacaagaaagagaggcctttgaaagaacatggtgggcaatttctgctgtaaagatgggcctccagattaataatgtagtagatggaaaggcatcattccagctcctaagagcgaaatatgaaaagaagactgctaataaaaagcagtctgagccctctgaagaatatctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgacctcgagggggggcccggtacccagcttttgttccctttagtgagggttaattgcgcgggaagtatttatcactaatcaagcacaagtaatacatgagaaacttttactacagcaagcacaatcctccaaaaaattttgtttttacaaaatccctggtgaacatgattggaagggacctactagggtgctgtggaagggtgatggtgcagtagtagttaatgatgaaggaaagggaataattgctgtaccattaaccaggactaagttactaataaaaccaaattgagtattgttgcaggaagcaagacccaactaccattgtcagctgtgtttcctgaggtctctaggaattgattacctcgatgcttcattaaggaagaagaataaacaaagactgaaggcaatccaacaaggaagacaacctcaatatttgttataaggtttgatatatgggagtatttggtaaaggggtaacatggtcagcatcgcattctatgggggaatcccagggggaatctcaacccctattacccaacagtcagaaaaatctaagtgtgaggagaacacaatgtttcaaccttattgttataataatgacagtaagaacagcatggcagaatcgaaggaagcaagagaccaagaaatgaacctgaaagaagaatctaaagaagaaaaaagaagaaatgactggtggaaaataggtatgtttctgttatgcttagcaggaactactggaggaatactttggtggtatgaaggactcccacagcaacattatatagggttggtggcgatagggggaagattaaacggatctggccaatcaaatgctatagaatgctggggttccttcccggggtgtagaccatttcaaaattacttcagttatgagaccaatagaagcatgcatatggataataatactgctacattattagaagctttaaccaatataactgctctataaataacaaaacagaattagaaacatggaagttagtaaagacttctggcataactcctttacctatttcttctgaagctaacactggactaattagacataagagagattttggtataagtgcaatagtggcagctattgtagccgctactgctattgctgctagcgctactatgtcttatgttgctctaactgaggttaacaaaataatggaagtacaaaatcatacttttgaggtagaaaatagtactctaaatggtatggatttaatagaacgacaaataaagatattatatgctatgattcttcaaacacatgcagatgttcaactgttaaaggaaagacaacaggtagaggagacatttaatttaattggatgtatagaaagaacacatgtattttgtcatactggtcatccctggaatatgtcatggggacatttaaatgagtcaacacaatgggatgactgggtaagcaaaatggaagatttaaatcaagagatactaactacacttcatggagccaggaacaatttggcacaatccatgataacattcaatacaccagatagtatagctcaatttggaaaagacctttggagtcatattggaaattggattcctggattgggagcttccattataaaatatatagtgatgtttttgcttatttatttgttactaacctcttcgcctaagatcctcagggccctctggaaggtgaccagtggtgcagggtcctccggcagtcgttacctgaagaaaaaattccatcacaaacatgcatcgcgagaagacacctgggaccaggcccaacacaacatacacctagcaggcgtgaccggtggatcaggggacaaatactacaagcagaagtactccaggaacgactggaatggagaatcagaggagtacaacaggcggccaaagagctgggtgaagtcaatcgaggcatttggagagagctatatttccgagaagaccaaaggggagatttctcagcctggggcggctatcaacgagcacaagaacggctctggggggaacaatcctcaccaagggtccttagacctggagattcgaagcgaaggaggaaacatttatgactgttgcattaaagcccaagaaggaactctcgctatcccttgctgtggatttcccttatggctattttggggactagtaattatagtaggacgcatagcaggctatggattacgtggactcgctgttataataaggatttgtattagaggcttaaatttgatatttgaaataatcagaaaaatgcttgattatattggaagagctttaaatcctggcacatctcatgtatcaatgcctcagtatgtttagaaaaacaaggggggaactgtggggtttttatgaggggttttataaatgattataagagtaaaaagaaagttgctgatgctctcataaccttgtataacccaaaggactagctcatgttgctaggcaactaaaccgcaataaccgcatttgtgacgcgagttccccattggtgacgcgttaacttcctgtttttacagtatataagtgcttgtattctgacaattgggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 73 - DNA, 517 bp, Artificial Sequence
  Description of Artificial Sequence Codon optimised EIAV REV
gaattcgccaccatggctgagagcaaggaggccagggatcaagagatgaacctcaaggaagagagcaaagaggagaagcgccgcaacgactggtggaagatcgacccacaaggccccctggagggggaccagtggtgccgcgtgctgagacagtccctgcccgaggagaagattcctagccagacctgcatcgccagaagacacctcggccccggtcccacccagcacacaccctccagaagggataggtggattaggggccagattttgcaagccgaggtcctccaagaaaggctggaatggagaattaggggcgtgcaacaagccgctaaagagctgggagaggtgaatcgcggcatctggagggagctctacttccgcgaggaccagaggggcgatttctccgcatggggaggctaccagagggcacaagaaaggctgtggggcgagcagagcagcccccgcgtcttgaggcccggagactccaaaagacgccgcaaacacctgtgaagtcgac